(19) United States
(12) Patent Application Publication
(10) Pub. No.: US 2025/0290937 A1
(43) Pub. Date: Sep. 18, 2025

(54) Methods and monitoring of treatment with lanifibranor

(71) Applicant: INVENTIVA, Daix (FR)

(72) Inventors: Louis H. GRIFFEL, Springfield, NJ (US); Martine BAUDIN, Daix (FR)

(73) Assignee: INVENTIVA, Daix (FR)

(21) Appl. No.: 19/080,283

(22) Filed: Mar. 14, 2025

Related U.S. Application Data
(60) Provisional application No. 63/565,738, filed on Mar. 15, 2024.
(60) Provisional application No. 63/665,078, filed on Jun. 27, 2024.

(30) Foreign Application Priority Data
Mar. 15, 2024 (WO) PCT/IB2024/000146

(51) Int. Cl. G01N 33/72; A61K 31/427; C12Q 1/44; C12Q 1/52; G01N 33/68; G01N 33/86

(52) U.S. Cl. CPC G01N 33/728; A61K 31/427; C12Q 1/44; C12Q 1/52; G01N 33/6857; G01N 33/86; G01N 2333/745; G01N 2333/9108; G01N 2333/91188; G01N 2333/916; G01N 2800/52

(57) ABSTRACT

The present disclosure relates to methods for identifying a subject as eligible for treatment with lanifibranor, methods for treating a subject suffering from liver disease comprising administering lanifibranor to said subject and monitoring a subject's risk of experiencing a side effect caused by lanifibranor treatment.

### FIELD OF THE DISCLOSURE

The present disclosure relates to methods for identifying a subject as eligible for treatment with lanifibranor, methods for treating a subject suffering from liver disease comprising administering lanifibranor to said subject and monitoring a subject's risk of experiencing a side effect caused by lanifibranor treatment.

### BACKGROUND OF THE DISCLOSURE

Liver diseases such liver fibrosis, liver steatosis, fatty liver disease, acute decompensation, acute on chronic liver failure, acute liver failure, decompensated cirrhosis, compensated cirrhosis, non-alcoholic fatty liver disease, non-alcoholic steatohepatitis, metabolic-associated fatty liver disease, metabolic-associated steatohepatitis, diabetes, primary sclerosing cholangitis, hepatocarcinoma and liver cirrhosis, are leading causes of morbidity and mortality. Typically, not all subjects who have a liver disease respond similarly well to a therapy. Health care can be improved by methods that enable matching a subject with a therapy to which the subject is most likely to respond. A major disadvantage to overcome is that no method exists to identify a subject who is likely to respond favorably to treatment with lanifibranor. Currently, the only means to select subjects for lanifibranor treatment is to observe the clinical course of disease during treatment. This potentially delays when a subject will receive effective treatment, thereby increasing morbidity risk and decreasing quality of life. Accordingly, there remains a need to identify one or more biomarkers, that are associated with disorders related to response to lanifibranor.

### SUMMARY OF THE DISCLOSURE

The present disclosure provides a method of identifying a subject as eligible for treatment with lanifibranor, comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker, and
d) selecting the subject for treatment with lanifibranor if:
   (i) the panel of autoimmune disease biomarkers is absent in the reference sample, and
   (ii) the subject reference level of at least one liver biomarker is equal or below than the reference level of the at least one liver biomarker.

The present disclosure also provides a method of screening a subject at risk of adverse side effect from treatment with lanifibranor, comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
wherein the subject is at risk of adverse side effect from treatment with lanifibranor if:
   (i) the panel of autoimmune disease biomarkers is present in the sample, and/or
   (ii) the subject reference level of at least one liver biomarker is higher than the reference level of the at least one liver biomarker.

The present disclosure also provides a method for reducing the risk or occurrence of adverse effect in a subject suffering from liver disease, the method comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) administering to said subject an effective amount of lanifibranor if:
   (i) the panel of autoimmune disease biomarkers is absent in the reference sample, and
   (ii) the subject reference level of at least one liver biomarker is below than the reference level of the at least one liver biomarker.

The present disclosure also provides a method for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of at least one liver biomarker in a baseline sample obtained from the subject; and
c) comparing the subject baseline level of the at least one liver biomarker to the corresponding upper normal limit of the at least one liver biomarker, to the reference level of the at least one liver biomarker and to the subject reference level of the at least one liver biomarker;
   wherein subject's risk of experiencing the adverse side effect when:
(i) the panel of autoimmune disease biomarkers is present in a baseline sample obtained from the subject, and/or
(ii) the subject baseline level of the at least one liver biomarker is higher than the reference level of said at least one liver biomarker; or
(iii) the subject baseline level of the at least one liver biomarker is higher than 300 U/L; or
(iv) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
(v) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L.

According to some embodiments, the panel of autoimmune disease biomarkers is indicative for predisposition to autoimmune disease. In some embodiments, the panel of autoimmune disease biomarkers comprises at least one autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

According to some embodiments, the at least one liver biomarker is selected from the group comprising: aspartate transaminase, alanine transaminase, alkaline phosphatase, gamma-glutamyl transferase, bilirubin, prothrombin time test with an international normalized ratio and a combination thereof.

According to some embodiments, the reference sample is a sample obtained from the subject prior to the first administration of lanifibranor treatment and wherein the baseline sample is a sample obtained from the same subject after administration of lanifibranor treatment. In some embodiments, the reference sample and the baseline sample are from the same source and are selected from saliva sample, hair sample, skin sample, tissue sample, sputum sample, blood sample, plasma sample, scrum sample, vitreal sample, cerebrospinal fluid sample, urine sample, sperm sample and cells sample.

According to some embodiments, the subject is suffering from a liver disease selected among: liver fibrosis, liver steatosis, fatty liver disease, acute decompensation, acute decompensation of cirrhosis, acute on chronic liver failure, acute liver failure, decompensated cirrhosis, compensated cirrhosis, non-alcoholic fatty liver disease, non-alcoholic steatohepatitis, metabolic-associated fatty liver disease, metabolic-associated steatohepatitis, diabetes, primary sclerosing cholangitis, hepatocarcinoma, non-alcoholic steatohepatitis with moderate to advanced liver fibrosis, metabolic-associated steatohepatitis with moderate to advanced liver fibrosis, non-cirrhotic non-alcoholic steatohepatitis with moderate to advanced liver fibrosis, non-cirrhotic metabolic-associated steatohepatitis with moderate to advanced liver fibrosis and liver cirrhosis.

According to some embodiments, the autoimmune disease is selected from the group comprising: itis, dermatomyositis, juvenile idiopathic arthritis, mixed connective tissue disease, primary biliary cholangitis, polymyositis, systemic autoimmune rheumatic disease, Sjögren syndrome, systemic lupus erythematosus, systemic sclerosis, undifferentiated connective tissue disease, autoimmune liver disease and autoimmune thyroid disease. In some embodiments, the autoimmune liver disease is selected from the group comprising: autoimmune hepatitis, autoimmune cholangitis, autoimmune cholangiopathy and overlap syndrome.

According to some embodiments, the effective amount of lanifibranor comprises from 0.5 to 1200 mg of lanifibranor once a day.

### DETAILED DESCRIPTION

The present disclosure may be understood more readily by reference to the following detailed description of the preferred embodiments of the disclosure. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the articles “a” and “an” refer to one or to more than one (i.e., to at least one) of the grammatical object of the article. By way of example, “an element” means one element or more than one element.

As used herein, the term “about xx” refers to a measurable value such as a parameter, an amount, a temporal duration, and the like, is meant to encompass variations of +/−10% or less, preferably +/−5% or less, more preferably +/−1% or less, even more preferably +/−0.5% or less, more preferably +/−0.05% or less, and still more preferably +/−0.04% or less, +/−0.03% or less, +/−0.02% or less, +/−0.01% or less of and from the specified value, insofar such variations are appropriate to perform in the disclosed disclosure. However, it is to be understood that the value to which “about” refers is itself also specifically disclosed. In other words, the term “about xx” includes the value “xx”.

As used herein, the expression “from xx to yy” includes the end points xx and yy of the range. As used herein, “biomarker” encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. In some aspects, therefore, a biomarker includes a protein or a fragment thereof or a nucleic acid or a fragment thereof.

The terms “level” and “amount” are used herein interchangeably to mean the concentration of biomarker present in a sample.

As used herein, the term “patient” or “subject” refers to a human individual or an animal different from a human. The patient is for example a human or an animal liable to have a liver disease or suffering from such a disease. The patient is advantageously a human being. The patient may be a child (human patient 18 years old or less) or an adult (human patient more than 18 years old). In some embodiments, the subject is a non-human animal including, but not limited to dog, cat, guinea pig, rabbit, rat, mouse, horse, cattle, bear, cow, ape, monkey, orangutan, and chimpanzee, and so on. In some embodiments, the subject is a human. In the context of the present disclosure the terms “patient” and “subject” are used interchangeably. In some embodiments, the subject of the present disclosure is suffering from a liver disease selected among: liver fibrosis, liver steatosis, fatty liver disease, acute decompensation, acute decompensation of cirrhosis, acute on chronic liver failure (ACLF), acute liver failure (ALF), decompensated cirrhosis, compensated cirrhosis, non-alcoholic fatty liver disease (NAFLD), non-alcoholic steatohepatitis (NASH), metabolic-associated fatty liver disease (MAFLD), metabolic-associated steatohepatitis (MASH), primary sclerosing cholangitis (PSC), hepatocarcinoma, non-alcoholic steatohepatitis with moderate to advanced liver fibrosis (consistent with stages F2 to F3 fibrosis), metabolic-associated steatohepatitis with moderate to advanced liver fibrosis (consistent with stages F2 to F3 fibrosis), non-cirrhotic non-alcoholic steatohepatitis with moderate to advanced liver fibrosis (consistent with stages F2 to F3 fibrosis), non-cirrhotic metabolic-associated steatohepatitis with moderate to advanced liver fibrosis (consistent with stages F2 to F3 fibrosis) and liver cirrhosis. As used herein, the term “acute decompensation” refers to an abrupt deterioration of liver function in patients with advanced chronic liver diseases, compensated cirrhosis or stable decompensated cirrhosis requiring immediate hospitalization. At hospital admission, patients with acute decompensation have multiple symptoms including, severe ascites, hepatic encephalopathy, variceal bleeding associated or not with sepsis and/or impaired renal function and/or coagulopathy and/or impaired cardiovascular function and/or impaired respiratory function. Acute decompensation is a life-threatening condition with an overall mortality rate of 11% at 28-Days. In some embodiments, “acute decompensation” refers to acute decompensation of cirrhosis. In some embodiments, the liver disease can be associated with diabetes, and in particular with type 2 diabetes. In some embodiments, the liver disease is associated with diabetes, and in particular with type 2 diabetes. Examples of liver disease associated with diabetes are: non-alcoholic fatty liver disease (NAFLD) associated with diabetes, non-alcoholic steatohepatitis (NASH) associated with diabetes, metabolic-associated fatty liver disease (MAFLD) associated with diabetes, metabolic-associated steatohepatitis (MASH) associated with diabetes, the list being not limitative.

The term “treat” or “treating” a liver disease as used herein means to administer a therapy according to the present disclosure to a subject having liver disease, or diagnosed with liver disease, to achieve at least one positive therapeutic effect, such as, for example, resolution of NASH and/or improvement of fibrosis and/or no fibrosis progression, reduction of lobular inflammation and/or reduction of hepatocyte ballooning and/or reduction of steatosis, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As used herein, the term “lanifibranor”, also called 1-(6-benzothiazolylsulfonyl)-5-chloro-1H-indole-2-butanoic acid is a pan-PPAR agonist with EC50s (as determined in J. Med. Chem. 2018, 61, 2246-2265) of 1.5, 0.87 and 0.21 UM for human PPARα, PPARδ and PPARγ, respectively. The chemical formula of lanifibranor is C₁₉H₁₅ClN₂O₄S₂, its molecular weight is of 434.92 and its CAS Number is 927961-18-0. Lanifibranor is described in example 117 of WO 2007/026097, where it is obtained as a pale-yellow powder having a melting point of 74-80° C., and has the following structure:

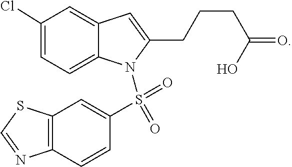 [SMILES: O=C(O)CCCc1cc2cc(Cl)ccc2n1S(=O)(=O)c1ccc2ncsc2c1]

In some embodiments, lanifibranor is in crystalline form. Examples of crystalline form of lanifibranor have been described in WO 2023/194339, in WO 2023/016319, in WO 2022/122014, in WO 2022/261410 or in WO 2022/258060, all incorporated herein by reference. The term “lanifibranor” also includes deuterated derivatives of lanifibranor. As used herein, “deuterated derivatives of lanifibranor” or “deuterated form of lanifibranor” refer to a compound having the structure of lanifibranor in which one or more hydrogen atoms is/are replaced by a deuterium atom. In particular, “deuterated derivatives of lanifibranor” or “deuterated form of lanifibranor” refer to a compound having the structure of lanifibranor in which at least one hydrogen atom is replaced by a deuterium atom. In some embodiments, a deuterated derivative of lanifibranor is a compound of formula (I):

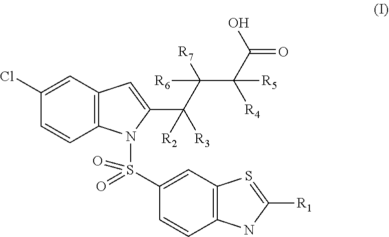

wherein at least one of the groups R₁ to R₇ is a deuterium (D) atom and the other groups R₁ to R₇ are hydrogen (H) atoms, as described in WO 2020/021215. In some embodiments, at least group R₁ is D. In some aspects at least one of the groups R₂ to R₇ is D, notably at least one of the groups R₂ and R₃ and/or at least one of the groups R₄ and Rs and/or at least one of the groups R₆ and Ry is D. In a preferred aspect each of R₂, R₃, R₄, R₅, R₆ and R₇ is D. Preferred compounds of formula (I) include 4-(1-(2-deuterio-1,3-benzothiazol-6-yl)sulfonyl)-5-chloro-1H-indol-2-yl)butanoic acid and 4-[1-(1,3-benzothiazol-6-ylsulfonyl)-5-chloro-indol-2-yl]-2,2,3,3,4,4-hexadeuteriobutanoic acid. In some embodiments, lanifibranor or a deuterated derivative thereof is in the form of one of its pharmaceutically acceptable salts or solvates.

As used herein, a “dosage”, an “amount”, an “effective dosage” or “effective amount” of drug, compound or pharmaceutical formulation is an amount sufficient to effect any one or more beneficial or desired, including biochemical, histological and/or behavioral symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. In reference to the treatment of liver disease, a therapeutically effective amount refers to that amount which has the effect of (1) resolution of NASH and/or improvement of fibrosis and/or no fibrosis progression, (2) reduction of lobular inflammation and/or reduction of hepatocyte ballooning and/or reduction of steatosis, (3) relieving to some extent (or, preferably, eliminating) one or more signs or symptoms associated with the liver disease, (4) decreasing the dose of other medications required to treat the disease, and/or (5) enhancing the effect of another medication, and/or delaying the progression of the disease of patients. An effective dosage can be administered in one or more administrations. For the purposes of this disclosure, an effective dosage of drug, compound, or pharmaceutical formulation is an amount sufficient to accomplish prophylactic or therapeutic treatment either directly or indirectly. As is understood in the clinical context, an effective dosage of drug, compound or pharmaceutical formulation may or may not be achieved in conjunction with another drug, compound or pharmaceutical formulation.

In some embodiments, an effective amount of lanifibranor or a pharmaceutically acceptable salt thereof is administered at a daily dosage of from about 0.5 mg to about 1,200 mg once a day, advantageously from about 50 mg to about 1,200 mg once a day, from about 100 mg to about 1,200 mg once a day, from about 200 mg to about 1,200 mg once a day, from about 300 mg to about 1,200 mg once a day, from about 400 mg to about 1,200 mg once a day, from about 500 mg to about 1,200 mg once a day, from about 600 mg to about 1,200 mg once a day, from about 700 mg to about 1,200 mg once a day, from about 800 mg to about 1,200 mg once a day, from about 900 mg to about 1,200 mg once a day, from about 1,000 mg to about 1,200 mg once a day, from about 1,100 mg to about 1,200 mg once a day. In an embodiment, an effective amount of lanifibranor or a pharmaceutically acceptable salt thereof is administered at a daily dosage of about 0.5 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, or about 1,200 mg once daily. In an embodiment, an effective amount of lanifibranor or a pharmaceutically acceptable salt thereof is administered at a daily dosage of 0.5 mg, 1 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, or 1,200 mg oncc daily. In an embodiment, an effective amount of lanifibranor or a pharmaceutically acceptable salt thereof is administered at a daily dosage of 400 mg once a day. In an embodiment, an effective amount of lanifibranor or a pharmaceutically acceptable salt thereof is administered at a daily dosage of 600 mg once a day. In an embodiment, an effective amount of lanifibranor or a pharmaceutically acceptable salt thereof is administered at a daily dosage of 800 mg once a day. In an embodiment, an effective amount of lanifibranor or a pharmaceutically acceptable salt thereof is administered at a daily dosage of 1,200 mg once a day. Dosage amounts provided herein refer to the dose of the free base form of lanifibranor or are calculated as the free base equivalent of an administered lanifibranor salt form. For example, a dosage or an effective amount of lanifibranor, such as 400 mg, 600 mg, 800 mg or 1,200 mg refers to the free base equivalent. This dosage regimen may be adjusted to provide the optimal therapeutic response. For example, the dose may be proportionally reduced or increased as indicated by the exigencies of the therapeutic situation.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As set forth above, the present method of identifying a subject as eligible for treatment with lanifibranor, comprising:
(i) determining the presence or the absence of a panel of autoimmune disease biomarkers in a reference sample obtained from the subject,
(ii) determining the subject reference level of at least one liver biomarker in a reference sample obtained from the subject,
(iii) comparing the subject reference level of the at least one liver biomarker to the reference level of the at least one liver biomarker, and
(iv) selecting the subject for treatment with lanifibranor if:
   (i) the panel of autoimmune disease biomarkers is absent in the reference sample, and
   (ii) the subject reference level of at least one liver biomarker is equal or below than the reference level of the at least one liver biomarker.

The method in accordance with the present disclosure includes a method which essentially consists of the aforementioned steps or a method which includes further steps. Moreover, the method of the present disclosure, preferably, is an ex vivo and more preferably an in vitro method. Moreover, it may comprise steps in addition to those explicitly mentioned above. For example, further steps may relate to the determination of further biomarkers and/or to sample pre-treatments or evaluation of the results obtained by the method. The method may be carried out manually or assisted by automation. Advantageously, step (a), (b) and/or (c) may in total or in part be assisted by automation, e.g., by a suitable robotic and sensory equipment for the determination in step (a) and (b) or a computer-implemented calculation in step (c). The term “identifying a patient” as used herein refers to using the information or data generated relating to the level of the panel of autoimmune disease biomarkers and to the level of at least one liver biomarker as referred to in the context of the present disclosure in a reference sample of a patient to identify or selecting the patient as more likely to benefit or less likely to benefit from a treatment with lanifibranor. Advantageously, a subject who is eligible for treatment with lanifibranor requires said treatment with lanifibranor, whereas a subject who is not eligible for treatment with lanifibranor does not require said treatment with lanifibranor. It is to be understood that a subject who is eligible for treatment with lanifibranor will benefit from the treatment with lanifibranor, whereas a subject who is not eligible for treatment with lanifibranor may not benefit from said treatment, e.g. may experience adverse side-effects from the treatment.

Thus, by identifying a subject as eligible for treatment with lanifibranor, it can be assessed whether said subject has to be treated with lanifibranor, or not. Accordingly, the present disclosure also relates to a method of identifying a subject a subject as eligible for treatment with lanifibranor, based on the steps set forth herein elsewhere.

As used herein, the term “sample” refers to any biological sample from a patient. Examples include, but are not limited to, saliva, hair, skin, tissue, sputum, blood, plasma, serum, vitreal, cerebrospinal fluid, urine, sperm and cells. In some embodiments, the sample is a plasma sample. In some embodiments, the sample is a serum sample. In some embodiments, the sample is a blood sample. According to the present disclosure, the term “reference sample obtained from a subject” is a sample obtained from said subject prior to the first administration of lanifibranor treatment. Advantageously, the reference sample is selected from saliva sample, hair sample, skin sample, tissue sample, sputum sample, blood sample, plasma sample, serum sample, vitreal sample, cerebrospinal fluid sample, urine sample, sperm sample and cells sample. In some embodiments, the reference sample obtained from a subject is a blood sample obtained from said subject prior to the first administration of lanifibranor treatment. Advantageously, the reference sample obtained from a subject is a serum sample obtained from said subject prior to the first administration of lanifibranor treatment. According to the present disclosure, the baseline sample obtained from subject is a sample obtained from the same subject after administration of lanifibranor treatment. The baseline sample is from the same source as the reference sample. The baseline sample and the reference sample are obtained from the same subject. For example, if the reference sample is a serum sample, the baseline sample is also a serum sample. In some embodiments, a baseline sample can be obtained at each visit of the subject.

As used herein, the term “subject reference level of at least one liver biomarker” or “patient reference level of at least one liver biomarker” is the level of the at least one liver biomarker in a reference sample obtained from a subject. Advantageously, the subject reference level of at least one liver biomarker is the level of the at least one liver biomarker in a reference sample obtained from a subject prior to the first administration of lanifibranor treatment.

As used herein, the term “subject baseline level of at least one liver biomarker” is the level of the at least one liver biomarker in a baseline sample obtained from a subject. Advantageously, the subject baseline level of at least one liver biomarker is the level of the at least one liver biomarker in a baseline sample obtained from a subject after administration of lanifibranor treatment.

According to the present disclosure, step a) of the method of identifying a subject as eligible for treatment with lanifibranor comprises determining the presence or the absence of a panel of autoimmune disease biomarkers in a reference sample obtained from the subject.

The term “determining” the level of biomarker (in particular a liver biomarker or an autoimmune disease biomarker) as referred to herein refers to the quantification of the biomarker, e.g. to measuring the level of the biomarker in the sample, employing appropriate methods of detection described elsewhere herein. The terms “measuring” and “determining” are used herein interchangeably. In an embodiment, the level of the biomarker is measured by contacting the sample with a detection agent that specifically binds to the respective biomarker, thereby forming a complex between the agent and said at least one autoimmune disease biomarker, detecting the level of complex formed, and thereby measuring the presence, the absence or the level of said biomarker.

As used herein, the phrase “determining the presence or absence” refers to assessing whether the biomarker, such as autoimmune disease biomarker, is detectable or undetectable in a sample using one or more detection techniques as described above. An autoimmune disease biomarker that is detected in a sample using a detection technique is considered “present”. An autoimmune disease biomarker that is not detected in a sample using a detection technique is considered “absent”. In some embodiments, negative result obtained by using one or more detection techniques as described above means that the autoimmune disease biomarker is considered “absent”. In some embodiments, positive result obtained by using one or more detection techniques as described above means that the autoimmune disease biomarker is considered “present”.

In some embodiments, the panel of autoimmune disease biomarkers is indicative for predisposition to autoimmune disease. Advantageously, the autoimmune disease biomarkers are autoantibodies, advantageously autoantibodies indicative for predisposition to autoimmune disease. In some embodiments, the autoimmune disease is selected from the group comprising: dermatomyositis, juvenile idiopathic arthritis, mixed connective tissue disease, primary biliary cholangitis, polymyositis, systemic autoimmune rheumatic disease, Sjögren syndrome, systemic lupus erythematosus, systemic sclerosis, undifferentiated connective tissue disease, autoimmune liver disease and autoimmune thyroid disease. In some embodiments, the autoimmune liver disease is selected from the group comprising: autoimmune hepatitis, autoimmune cholangitis, autoimmune cholangiopathy and overlap syndrome. In some embodiments, the panel of autoimmune disease biomarkers is indicative for predisposition to autoimmune liver disease and autoimmune thyroid disease.

In some embodiments, step a) of the method of identifying a subject as eligible for treatment with lanifibranor comprises determining the presence or the absence of a panel of autoimmune disease biomarkers in a reference sample obtained from the subject, wherein the panel of autoimmune disease biomarkers comprises autoantibodies, advantageously autoantibodies indicative for predisposition to autoimmune disease.

In some embodiments, the panel of autoimmune disease biomarkers comprises at least one autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, anti-liver cytosol type 1 antibody and a combination thereof.

Advantageously, the panel of autoimmune disease biomarkers is a combination of at least two autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, step a) of the method of identifying a subject as eligible for treatment with lanifibranor comprises determining the presence or the absence of a panel of autoimmune disease biomarkers in a reference sample obtained from the subject, wherein the panel of autoimmune disease biomarkers is a combination of at least two autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of at least two, at least three, at least four, at least five, at least six, at least seven or at least eight autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of two autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of three autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of four autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of five autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of six autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of seven autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers used in step a) comprises a combination of eight autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the panel of autoimmune disease biomarkers comprises a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody; In some embodiments, the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, step a) of the method of identifying a subject as eligible for treatment with lanifibranor comprises determining the presence or the absence of a panel of autoimmune disease biomarkers in a reference sample obtained from the subject, wherein the panel of autoimmune disease biomarkers used in step a) comprises a combination of at least two, at least three, at least four, at least five, at least six, at least seven or at least eight autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, step a) of the method of identifying a subject as eligible for treatment with lanifibranor comprises determining the presence or the absence of a panel of autoimmune disease biomarkers in a reference sample obtained from the subject, wherein the panel of autoimmune disease biomarkers used in step a) comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, step a) of the method of identifying a subject as eligible for treatment with lanifibranor comprises determining the presence or the absence of a panel of autoimmune disease biomarkers in a reference sample obtained from the subject, wherein the panel of autoimmune disease biomarkers used in step a) is a the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the anti-nuclear antibody comprises a panel of different antibodies selected from the group comprising: anti-double-stranded DNA (dsDNA) antibody, anti-histone antibody, anti-chromatin antibody, anti-Scl-70 antibody, anti-smith antibody, anti-Sm/RNP antibody, anti-Jo-1 antibody, anti-centromeric protein antibody, anti-SSA-52 antibody, anti-SSA-60 antibody, anti-SSB antibody, anti-RNA polymerase III antibody, anti-Ku antibody, anti-CENP-A antibody, anti-CENP-B antibody, anti-fibrillarin antibody, anti-PM/Scl-100 antibody, anti-Th/To antibody, anti-Sp-100 antibody, anti-PML antibody, anti-NXP-2 antibody, anti-MORC3 antibody, anti-MJ antibody and a combination thereof. According to the present disclosure, step b) of the method of identifying a subject as eligible for treatment with lanifibranor comprises determining the subject reference level of at least one liver biomarker in a reference sample obtained from the subject.

In some embodiments, the at least one liver biomarker is selected from the group comprising: aspartate transaminase (AST), alanine transaminase (ALT), alkaline phosphatase (ALP), gamma-glutamyl transferase (GGT), bilirubin, prothrombin time test with an international normalized ratio (PR/INR) and a combination thereof. In some embodiments, the term “bilirubin” refers to total bilirubin and/or direct bilirubin.

In some embodiments, the at least one liver biomarker used in step b) is aspartate transaminasc.

In some embodiments, the at least one liver biomarker used in step b) is alanine transaminase.

In some embodiments, the at least one liver biomarker used in step a) is alkaline phosphatase.

In some embodiments, the at least one liver biomarker used in step b) is gamma-glutamyl transferase. In some embodiments, the at least one liver biomarker used in step b) is bilirubin.

In some embodiments, the at least one liver biomarker used in step b) is prothrombin time test with an international normalized ratio.

In some embodiments, the at least one liver biomarker used in step a) is a combination of at least two, at least three, at least four, at least five, or at least six liver biomarkers selected from: aspartate transaminase, alanine transaminase, alkaline phosphatase, gamma-glutamyl transferase, bilirubin, and prothrombin time test with an international normalized ratio. In some embodiments, the at least one liver biomarker used in step a) is a combination of liver biomarkers comprises aspartate transaminase and alanine transaminase. In some embodiments, the at least one liver biomarker used in step a) is a combination of liver biomarkers comprises aspartate transaminase, alanine transaminase and alkaline phosphatase. In some embodiments, the at least one liver biomarker used in step a) is a combination of liver biomarkers comprises aspartate transaminase, alanine transaminase, alkaline phosphatase and gamma-glutamyl transferase. In some embodiments, the at least one liver biomarker used in step a) is a combination of liver biomarkers comprises aspartate transaminase, alanine transaminase, alkaline phosphatase, gamma-glutamyl transferase and bilirubin. In some embodiments, the at least one liver biomarker used in step a) is a combination of liver biomarkers comprises aspartate transaminase, alanine transaminase and bilirubin. In some embodiments, the at least one liver biomarker used in step a) is a combination of liver biomarkers comprises aspartate transaminase, alanine transaminase, gamma-glutamyl transferase and bilirubin. In some embodiments, the at least one liver biomarker used in step a) is a combination of liver biomarkers comprises aspartate transaminase, alanine transaminase, alkaline phosphatase, gamma-glutamyl transferase, bilirubin, and prothrombin time test with an international normalized ratio.

In some embodiments, the at least one liver biomarker used in step a) is a combination of liver biomarkers consisting of aspartate transaminase, alanine transaminase, alkaline phosphatase, gamma-glutamyl transferase, bilirubin, and prothrombin time test with an international normalized ratio.

The biomarker as referred to herein can be detected using methods generally known in the art. Methods of detection generally encompass methods to quantify the level of a biomarker in the sample (quantitative method). It is generally known to the skilled artisan which of the following methods are suitable for qualitative and/or for quantitative detection of a biomarker. Biomarkers generally can be measured and detected through a variety of assays, methods and detection systems known to one of skill in the art. Various methods include but are not limited to immunoassays like ELISAs, RIAs, quantitative chemiluminescent immunoassay, fluorescence-based immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance. Said methods can comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays, enzymatic Cobalt Binding Assay and latex agglutination assays. For the detection of biomarker proteins as referred to herein a wide range of immunoassay techniques using such an assay format are available. These include both single-site and two-site or “sandwich” assays of the non-competitive types, as well as in the traditional competitive binding assays. These assays also include direct binding of a labeled antibody to a target autoimmune disease biomarker. Sandwich assays are among the most useful and commonly used immunoassays. Methods for measuring electrochemiluminescent phenomena are well-known. Such methods make use of the ability of special metal complexes to achieve, by means of oxidation, an excited state from which they decay to ground state, emitting electrochemiluminescence. Biomarkers can also be detected by generally known methods including magnetic resonance spectroscopy (NMR spectroscopy), Gas chromatography-mass spectrometry (GC-MS), Liquid chromatography-mass spectrometry (LC-MS), High and ultra-HPLC HPLC such as reverse phase HPLC, for example, ion-pairing HPLC with dual UV-wavelength detection, capillary electrophoresis with laser-induced fluorescence detection, anion exchange chromatography and fluorescent detection, thin layer chromatography. In accordance with the present disclosure, determining the presence, absence or the level of a biomarker can be achieved by all known means for determining the presence, absence or the level of a peptide in a sample. Examples of such means include immunoassay devices and methods which may utilize labeled molecules in various sandwich, competition, or other assay formats. These assays will develop a signal which is indicative for the presence or absence of the biomarker. Moreover, the signal strength can be correlated directly or indirectly (e.g. reverse-proportional) to the level of biomarker present in a sample. Further suitable methods comprise measuring a physical or chemical property specific for the biomarker such as its precise molecular mass or NMR spectrum. These methods may comprise biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, enzymatic Cobalt Binding Assay and latex agglutination assays.

In some embodiments, the presence or absence of the panel of autoimmune disease biomarkers is determined using immunoassay techniques. In some embodiments, the presence or absence of anti-thyroid peroxidase antibody is determined using quantitative chemiluminescent immunoassay. In some embodiments, the presence or absence of anti-thyroglobulin antibody is determined using quantitative chemiluminescent immunoassay. In some embodiments, the presence or absence of anti-thyroid-stimulating hormone (TSH) receptor antibody is determined using quantitative electrochemiluminescent immunoassay (ECLIA). In some embodiments, the presence or absence of anti-nuclear antibody is determined using ELISA.

Advantageously, the presence or absence of anti-nuclear antibody is determining at a dilution of 1:320 or greater. In some embodiments, the presence or absence of anti-mitochondrial antibody is determined using ELISA. In some embodiments, the presence or absence of anti-smooth muscle antibody is determined using ELISA. Advantageously, the presence or absence of anti-nuclear antibody is determining at a dilution of 1:320 or greater. In some embodiments, the presence or absence of anti-liver kidney microsomal type 1 antibody is determined using ELISA. In some embodiments, the presence or absence of anti-liver cytosol type 1 antibody is determined using qualitative immunoblot.

In some embodiments, the presence, absence or level of at least one liver biomarker is determined using colorimetry method, such as enzymatic colorimetric method or standard method of International Federation of Clinical Chemistry (IFCC), with or without pyridoxal phosphate activation. In particular, the presence, absence or level of aspartate transaminase (AST) and alanine transaminase (ALT) is determined using the standard method of International Federation of Clinical Chemistry (IFCC), with or without pyridoxal phosphate activation. In particular, the presence, absence or level of alkaline phosphatase (ALP), gamma-glutamyl transferase (GGT) and bilirubin (total bilirubin or direct bilirubin) is determined by using colorimetry method.

In some embodiments, the prothrombin time (PT) test measures how long it takes for a clot to form in a sample. The result of the prothrombin time test is expressed as an international normalized ratio (INR) to standardize the results. According to the present disclosure, the skilled person in the art is able to select the suitable method among the well-known methods for determining the level of PT/INR.

According to the present disclosure, step c) of the method comprises comparing the subject reference level of the at least one liver biomarker to the reference level of the at least one liver biomarker.

As used herein, “upper normal limit” (UNL) refers to the level of a liver biomarker biomarker measured in a sample of a healthy subject. According to the present disclosure, the upper normal limit of aspartate transaminase (AST) is 32 U/L for women and 40 U/L for men, as measured according to the standard method of International Federation of Clinical Chemistry (IFCC) with or without pyridoxal phosphate activation, using for example the Roche Cobas instrument. According to the present disclosure, the upper normal limit of alanine transaminase (ALT) is 33 U/L for women and 41 U/L for men, as measured according to the standard method of IFCC with or without pyridoxal phosphate activation, using for example the Roche Cobas instrument. According to the present disclosure, the upper normal limit of alkaline phosphatase (ALP) is 104 U/L for women and 129 U/L for men, as measured according to the standard method of IFCC by the enzymatic colorimetric method using for example the Roche Cobas instrument. According to the present disclosure, the upper normal limit of gamma-glutamyl transferase (GGT) is 36 U/L for women and 61 U/L for men, as measured by enzymatic-kinetic Szasz method. According to the present disclosure, the upper normal limit of total bilirubin is 20.5 μmol/L, as measured by enzymatic-kinetic Szasz method using for example the Roche Cobas instrument. According to the present disclosure, the upper normal limit of direct bilirubin is 5.1 μmol/L, as measured by DPD (N,N-diethyl-p-phenylenediamine) colorimetric method using for example the Roche Cobas instrument. According to the present disclosure, the upper normal limit of the PT/INR ratio is 1.20, as calculated using Stago STA instrument.

According to some embodiments, the reference level of aspartate transaminase is five times the corresponding upper normal limit. In particular, the reference level of aspartate transaminase is 160 U/L for women and 200 U/L for men. According to some embodiments, the reference level of alanine transaminase is five times the corresponding upper normal limit. In particular, the reference level of alanine transaminase is 165 U/L for women and 205 U/L for men. According to some embodiments, the reference level of alkaline phosphatase is two times the corresponding upper normal limit. In particular, the reference level of alkaline phosphatase is 208 U/L for women and 258 U/L for men. According to some embodiments, the reference level of gamma-glutamyl transferase is two times the corresponding upper normal limit. In particular, the reference level of gamma-glutamyl transferase is 72 U/L for women and 122 U/L for men. According to some embodiments, the reference level of total bilirubin is 1.25 times the corresponding upper normal limit. In particular, the reference level of total bilirubin is 25.6 μmol/L. According to some embodiments, the reference level of direct bilirubin is 1.51 times the corresponding upper normal limit. In particular, the reference level of direct bilirubin is 7.7 μmol/L. According to some embodiments, the reference level of PT/INR ratio is 1.08 times the corresponding upper normal limit. In particular, the reference level of PT/INR ratio is 1.30. According to the present disclosure, step d) of the method comprises selecting the subject for treatment with lanifibranor if:
(i) the panel of autoimmune disease biomarker is absent in the reference sample, and
(ii) the subject reference level of at least one liver biomarker is equal or below than the reference level of said at least one liver biomarker.

In some embodiments, the panel of autoimmune disease biomarker comprises a combination of at least two, at least three, at least four, at least five, at least six, at least seven and at least eight autoimmune disease biomarkers, selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

Advantageously, at least two, at least three, at least four, at least five, at least six, at least seven and at least eight autoimmune disease biomarkers, selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody are absent in the reference sample.

In some embodiments, a subject is selected for treatment with lanifibranor if the panel of autoimmune disease biomarker is absent in the reference sample and if the subject reference level of at least one liver biomarker is equal or below than the reference level of said at least one liver biomarker.

In some embodiments, a subject is selected for treatment with lanifibranor if the panel of autoimmune disease biomarker is absent in the reference sample and if the subject reference level of at least two liver biomarkers is equal or below than the reference level of said at least two liver biomarkers.

In some embodiments, a subject is selected for treatment with lanifibranor if the panel of autoimmune disease biomarker is absent in the reference sample and if the subject reference level of at least three liver biomarkers is equal or below than the reference level of said at least three liver biomarkers.

In some embodiments, a subject is selected for treatment with lanifibranor if the panel of autoimmune disease biomarker is absent in the reference sample and if the subject reference level of at least four liver biomarkers is equal or below than the reference level of said at least four liver biomarkers.

In some embodiments, a subject is selected for treatment with lanifibranor if the panel of autoimmune disease biomarker is absent in the reference sample and if the subject reference level of at least five liver biomarkers is equal or below than the reference level of said at least five liver biomarkers.

In some embodiments, a subject is selected for treatment with lanifibranor if the panel of autoimmune disease biomarker is absent in the reference sample and if the subject reference level of at least six liver biomarkers is equal or below than the reference level of said at least six liver biomarkers.

In some embodiments, a subject is selected for treatment with lanifibranor if the panel of autoimmune disease biomarkers is absent in the reference sample and if the subject reference level of the six liver biomarkers is equal or below than the reference level of said six liver biomarkers.

In some embodiments, a subject is selected for treatment with lanifibranor:
if the panel of autoimmune disease biomarker is absent in the reference sample, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and
if the subject reference level of the six liver biomarkers is equal or below than the reference level of said six liver biomarkers, wherein the six liver biomarkers are selected from: aspartate transaminase (AST), alanine transaminase (ALT), alkaline phosphatase (ALP), gamma-glutamyl transferase (GGT), bilirubin and prothrombin time test with an international normalized ratio (PR/INR).

In some embodiments, a subject is selected for treatment with lanifibranor:
if the panel of autoimmune disease biomarker is absent in the reference sample, and
if the subject reference level of aspartate transaminase is equal or below than five times the corresponding upper normal limit, and
if the subject reference level of alanine transaminase is equal or below than five times the corresponding upper normal limit, and
if the subject reference level of alkaline phosphatase is equal or below than two times the corresponding upper normal limit, and
if the subject reference level of gamma-glutamyl transferase is equal or below than two times the corresponding upper normal limit, and
if the subject reference level of total bilirubin is equal or below than 1.25 times the corresponding upper normal limit, and
if the subject reference level of direct bilirubin is equal or below than 1.51 times the corresponding upper normal limit, and
if the subject reference level of PT/INR ratio is equal or below than 1.08 times the corresponding upper normal limit.

In some embodiments, a subject is selected for treatment with lanifibranor:
if the panel of autoimmune disease biomarker is absent in the reference sample, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and
if the subject reference level of aspartate transaminase is equal or below than five times the corresponding upper normal limit, advantageously if the subject reference level of aspartate transaminase is equal or below than 160 U/L for women and 200 U/L for men, and
if the subject reference level of alanine transaminase is equal or below than five times the corresponding upper normal limit, advantageously if the subject reference level of alanine transaminase is equal or below than 165 U/L for women and 205 U/L for men, and
if the subject reference level of alkaline phosphatase is equal or below than two times the corresponding upper normal limit, advantageously if the subject reference level of alkaline phosphatase is equal or below than 208 U/L for women and 258 U/L for men, and
if the subject reference level of gamma-glutamyl transferase is equal or below than two times the corresponding upper normal limit, advantageously if the subject reference level of gamma-glutamyl transferase is equal or below than 72 U/L for women and 122 U/L for men, and
if the subject reference level of total bilirubin is equal or below than 1.25 times the corresponding upper normal limit, advantageously if the subject reference level of total bilirubin is equal or below than 25.6 μmol/L, and
if the subject reference level of direct bilirubin in the reference sample is equal or below than 1.51 times the corresponding upper normal limit, advantageously if the subject reference level of direct bilirubin in the reference sample is equal or below than 7.7 μmol/L, and
if the subject reference level of PT/INR ratio is equal or below than 1.08 times the corresponding upper normal limit, advantageously if the subject reference level of PT/INR ratio is equal or below than 1.30.

In some embodiments, the method of identifying a subject as eligible for treatment with lanifibranor further comprises prior to step a), a step of obtaining a reference sample from the subject.

In some embodiments, the method of identifying a subject as eligible for treatment with lanifibranor further comprises administering an effective amount of lanifibranor to the subject selected for treatment with lanifibranor after step d). In some embodiments, the effective amount of lanifibranor comprises from 0.5 to 1200 mg of lanifibranor once a day, as defined above.

In some embodiments, the present disclosure relates to a method of identifying a subject as eligible for treatment with lanifibranor, comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker, and
d) selecting the subject for treatment with lanifibranor if:
   (i) the panel of autoimmune disease biomarkers is absent in the reference sample, and
   (ii) the subject reference level of at least one liver is equal or below than the reference level of the at least one liver biomarker;
e) administering an effective amount of lanifibranor to the subject selected for treatment with lanifibranor.

In some embodiments, the present disclosure relates to a method of identifying a subject as eligible for treatment with lanifibranor, comprising:
a) obtaining a reference sample from the subject,
b) determining the presence or the absence of a panel of autoimmune disease biomarkers in a reference sample obtained from the subject,
c) determining the subject reference level of at least one liver biomarker in a reference sample obtained from the subject,
d) comparing the subject reference level of the at least one liver biomarker to the reference level of the at least one liver biomarker, and
e) selecting the subject for treatment with lanifibranor if:
   (iii) the panel of autoimmune disease biomarkers is absent in the reference sample, and
   (iv) the subject reference level of at least one liver biomarker is equal or below than the reference level of the at least one liver biomarker;
f) administering an effective amount of lanifibranor to the subject selected for treatment with lanifibranor.

In another aspect, the present disclosure relates to a method of treating a subject with lanifibranor, comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker, and
d) administering lanifibranor to the patient if the patient has:
   (i) the panel of autoimmune disease biomarkers is absent in the reference sample, and
   (ii) the subject reference level of at least one liver biomarker is equal or below than the reference level of the at least one liver biomarker.

According to the present disclosure, steps a), b) and c) are performed as defined above.

According to the present disclosure, step d) of the method comprises administering lanifibranor to the patient if the patient has:
(i) absence of the panel of autoimmune disease biomarkers in the reference sample, and
(ii) the subject reference level of at least one liver biomarker is equal or below than the reference level of said at least one liver biomarker.

In some embodiments, the panel of autoimmune disease biomarkers comprises a combination of at least two, at least three, at least four, at least five, at least six, at least seven and at least eight autoimmune disease biomarkers, selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

Advantageously, at least two, at least three, at least four, at least five, at least six, at least seven and at least eight autoimmune disease biomarkers, selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody are absent in the reference sample.

In some embodiments, lanifibranor is administered to the subject, if the panel of autoimmune disease biomarkers is absent in the subject's reference sample and if the subject reference level of at least one liver biomarker is equal or below than the reference level of said at least one liver biomarker.

In some embodiments, lanifibranor is administered to the subject, if the panel of autoimmune disease biomarkers is absent in the subject's reference sample and if the subject reference level of at least two liver biomarkers is equal or below than the reference level of said at least two liver biomarkers.

In some embodiments, lanifibranor is administered to the subject, if the panel of autoimmune disease biomarkers is absent in the subject's reference sample and if the subject reference level of at least three liver biomarkers is equal or below than the reference level of said at least three liver biomarkers.

In some embodiments, lanifibranor is administered to the subject, if the panel of autoimmune disease biomarkers is absent in the subject's reference sample and if the subject reference level of at least four liver biomarkers is equal or below than the reference level of said at least four liver biomarkers.

In some embodiments, lanifibranor is administered to the subject, if the panel of autoimmune disease biomarkers is absent in the subject's reference sample and if the subject reference level of at least five liver biomarkers is equal or below than the reference level of said at least five liver biomarkers.

In some embodiments, lanifibranor is administered to the subject, if the panel of autoimmune disease biomarkers is absent in the subject's reference sample and if the subject reference level of at least six liver biomarkers is equal or below than the reference level of said at least six liver biomarkers.

In some embodiments, lanifibranor is administered to the subject, if the panel of autoimmune disease biomarkers is absent in the subject's reference sample and if the subject reference level of the six liver biomarkers is equal or below than the reference level of said six liver biomarkers. In some embodiments, lanifibranor is administered to the subject, if:
the panel of autoimmune disease biomarkers is absent in the subject's reference sample, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and
the subject reference level of the six liver biomarkers is equal or below than the reference level of said six liver biomarkers, wherein the six liver biomarkers are selected from: aspartate transaminase (AST), alanine transaminase (ALT), alkaline phosphatase (ALP), gamma-glutamyl transferase (GGT), bilirubin and prothrombin time test with an international normalized ratio (PR/INR).

In some embodiments, lanifibranor is administered to the subject, if:
the panel of autoimmune disease biomarkers is absent in the subject's reference sample, and
the subject reference level of aspartate transaminase is equal or below than the reference level of aspartate transaminase, and
the subject reference level of alanine transaminase in the reference sample is equal or below than the reference level of alanine transaminase, and
the subject reference level of alkaline phosphatase is equal or below than the reference level of alkaline phosphatase, and
the subject reference level of gamma-glutamyl transferase is equal or below than the reference level of gamma-glutamyl transferase, and
the subject reference level of total bilirubin in the reference sample is equal or below than the reference level of total bilirubin, and
the subject reference level of direct bilirubin in the reference sample is equal or below than the reference level of direct bilirubin, and
the subject reference level of PT/INR ratio in the reference sample is equal or below than the reference level of PT/INR ratio.

In some embodiments, lanifibranor is administered to the subject, if:
the panel of autoimmune disease biomarkers is absent in the subject's reference sample, and
the subject reference level of aspartate transaminase sample is equal or below than five times the corresponding upper normal limit, and
the subject reference level of alanine transaminase is equal or below than five times the corresponding upper normal limit, and
the subject reference level of alkaline phosphatase is equal or below than two times the corresponding upper normal limit, and
the subject reference level of gamma-glutamyl transferase is equal or below than two times the corresponding upper normal limit, and
the subject reference level of total bilirubin is equal or below than 1.25 times the corresponding upper normal limit, and
the subject reference level of direct bilirubin is equal or below than 1.51 times the corresponding upper normal limit, and
the subject reference level of PT/INR ratio is equal or below than 1.08 times the corresponding upper normal limit.

In some embodiments, lanifibranor is administered to the subject, if:
the panel of autoimmune disease biomarkers is absent in the subject's reference sample, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and
the subject reference level of aspartate transaminase is equal or below than five times the corresponding upper normal limit, advantageously if the subject reference level of aspartate transaminase is equal or below than 160 U/L for women and 200 U/L for men, and
the subject reference level of alanine transaminase is equal or below than five times the corresponding upper normal limit, advantageously if the subject reference level of alanine transaminase is equal or below than 165 U/L for women and 205 U/L for men, and
the subject reference level of alkaline phosphatase is equal or below than two times the corresponding upper normal limit, advantageously if the subject reference level of alkaline phosphatase is equal or below than 208 U/L for women and 258 U/L for men, and
the subject reference level of gamma-glutamyl transferase is equal or below than two times the corresponding upper normal limit, advantageously if the subject reference level of gamma-glutamyl transferase is equal or below than 72 U/L for women and 122 U/L for men, and
the subject reference level of total bilirubin is equal or below than 1.25 times the corresponding upper normal limit, advantageously if the subject reference level of total bilirubin is equal or below than 25.6 μmol/L, and
the subject reference level of direct bilirubin is equal or below than 1.51 times the corresponding upper normal limit, advantageously if the subject reference level of direct bilirubin is equal or below than 7.7 μmol/L, and
the subject reference level of PT/INR ratio is equal or below than 1.08 times the corresponding upper normal limit, advantageously if the subject reference level of PT/INR ratio is equal or below than 1.30.

In some embodiments, the method of identifying a subject as eligible for treatment with lanifibranor further comprises prior to step a), a step of obtaining a reference sample from the subject.

In some embodiments, the method of identifying a subject as eligible for treatment with lanifibranor further comprises administering an effective amount of lanifibranor to the subject selected for treatment with lanifibranor after step d). In some embodiments, the effective amount of lanifibranor comprises from 0.5 to 1200 mg of lanifibranor once a day.

In another aspect, the present disclosure relates to a method of screening a subject at risk of adverse side effect from treatment with lanifibranor, comprising:
a. determining the presence or the absence of a panel of autoimmune disease biomarkers in a reference sample obtained from the subject,
b. determining the subject reference level of at least one liver biomarker in a reference sample obtained from the subject,
C. comparing the subject reference level of the at least one liver biomarker to the reference level of the at least one liver biomarker,
   wherein the subject is at risk of adverse side effect from treatment with lanifibranor if:
(i) the panel of autoimmune disease biomarkers is present in the reference sample, and/or
(ii) the subject reference level of at least one liver biomarker is higher than the reference level of the at least one liver biomarker.

In some embodiments, the method of screening a subject at risk of adverse side effect from treatment with lanifibranor further comprises prior to step a), a step of obtaining a reference sample from the subject.

In some embodiments, the method of screening a subject at risk of adverse side effect from treatment with lanifibranor further comprises not administering lanifibranor to said subject.

Thus, if the subject is at risk of adverse side effect from treatment with lanifibranor, lanifibranor is contraindicated for said subject and should not be administered or given to said subject. If the subject is at risk of adverse side effect from treatment with lanifibranor, lanifibranor should not be used for treating said subject.

In another aspect, the present disclosure relates to a method of screening a subject at risk of adverse side effect from treatment with lanifibranor, comprising:
a) determining the presence or the absence of a panel of autoimmune disease biomarkers in a reference sample obtained from the subject;
b) determining the subject reference level of at least one liver biomarker in a reference sample obtained from the subject;
c) comparing the subject reference level of the at least one liver biomarker to the reference level of the at least one liver biomarker;
d) wherein the subject is at risk of adverse side effect from treatment with lanifibranor if the panel of autoimmune disease biomarkers is present in the reference sample, and/or the subject reference level of at least one liver biomarker is higher than the reference level of the at least one liver biomarker; and
e) not administering lanifibranor to said subject.

In some embodiments, the panel of autoimmune disease biomarker comprises a combination of at least two, at least three, at least four, at least five, at least six, at least seven and at least eight autoimmune disease biomarkers, selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody. In some embodiments, the panel of autoimmune disease biomarker comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody. In some embodiments, the anti-nuclear antibody comprises a panel of different antibodies selected from the group comprising: anti-double-stranded DNA (dsDNA) antibody, anti-histone antibody, anti-chromatin antibody, anti-Scl-70 antibody, anti-smith antibody, anti-Sm/RNP antibody, anti-Jo-1 antibody, anti-centromeric protein antibody, anti-SSA-52 antibody, anti-SSA-60 antibody, anti-SSB antibody, anti-RNA polymerase III antibody, anti-Ku antibody, anti-CENP-A antibody, anti-CENP-B antibody, anti-fibrillarin antibody, anti-PM/Scl-100 antibody, anti-Th/To antibody, anti-Sp-100 antibody, anti-PML antibody, anti-NXP-2 antibody, anti-MORC3 antibody, anti-MJ antibody and a combination thereof.

According to the present disclosure, steps a), b) and c) are performed as defined above. In some embodiments, the subject is at risk of adverse side effect from treatment with lanifibranor if:
the panel of autoimmune disease biomarker is present in the reference sample, and/or
the subject reference level of at least two liver biomarkers is higher than the reference level of said at least two liver biomarkers.

In some embodiments, the subject is at risk of adverse side effect from treatment with lanifibranor if:
the panel of autoimmune disease biomarker is present in the reference sample, and/or
the subject reference level of at least three liver biomarkers is higher than the reference level of said at least three liver biomarkers.

In some embodiments, the subject is at risk of adverse side effect from treatment with lanifibranor if:
the panel of autoimmune disease biomarker is present in the reference sample and/or
the subject reference level of at least four liver biomarkers is higher than the reference level of said at least four liver biomarkers.

In some embodiments, the subject is at risk of adverse side effect from treatment with lanifibranor if:
the panel of autoimmune disease biomarker is present in the reference sample, and/or
the subject reference level of at least five liver biomarkers is higher than the reference level of said at least five liver biomarkers.

In some embodiments, the subject is at risk of adverse side effect from treatment with lanifibranor:
the panel of autoimmune disease biomarker is present in the reference sample, and/or
the subject reference level of at least six liver biomarkers is higher than the reference level of said at least six liver biomarkers.

In some embodiments, the at least one liver biomarker is selected from the group comprising: aspartate transaminase, alanine transaminase, alkaline phosphatase, gamma-glutamyl transferase, bilirubin, prothrombin time test with an international normalized ratio and a combination thereof.

In some embodiments, the subject is at risk of adverse side effect from treatment with lanifibranor:
if the panel of autoimmune disease biomarker is present in the reference sample, wherein the panel of autoimmune biomarkers comprises at least one, at least two, at least three, at least four, at least five, at least six, at least seven or at least eight autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and/or
if the subject reference level of at least one liver biomarker is higher than the reference level of said at least one liver biomarker, wherein the at least one liver biomarker is selected from: aspartate transaminase (AST), alanine transaminase (ALT), alkaline phosphatase (ALP), gamma-glutamyl transferase (GGT), bilirubin and prothrombin time test with an international normalized ratio (PR/INR).

In some embodiments, the subject is at risk of adverse side effect from treatment with lanifibranor:
if the panel of autoimmune disease biomarker is present in the reference sample, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and/or
if the subject reference level of at least one liver biomarker is higher than the reference level of said at least one liver biomarker, wherein the at least one liver biomarker is selected from: aspartate transaminase (AST), alanine transaminase (ALT), alkaline phosphatase (ALP), gamma-glutamyl transferase (GGT), bilirubin and prothrombin time test with an international normalized ratio (PR/INR).

In some embodiments, the subject is at risk of adverse side effect from treatment with lanifibranor if the panel of autoimmune disease biomarker is present in the reference sample. Advantageously, the panel of autoimmune disease biomarker comprises at least one, at least two, at least three, at least four, at least five, at least six, at least seven or at least eight autoimmune disease biomarkers selected from: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody. In some embodiments, the panel of autoimmune disease biomarker comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the subject is at risk of adverse side effect from treatment with lanifibranor if the subject reference level of aspartate transaminase is higher than at least 5 times or more the corresponding upper normal limit. In some embodiments, the subject is at risk of adverse side effect from treatment with lanifibranor if the subject reference level of aspartate transaminase is higher than at least 5.5 times, at least 6 times, at least 6.5 times, at least 7 times, at least 7.5 times, at least 8 times, at least 8.5 times, at least 9 times, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more the corresponding upper normal limit.

In some embodiments, the subject is at risk of adverse side effect from treatment with lanifibranor if the subject reference level of alanine transaminase and the subject reference level of alanine transaminase are higher than at least 5 times or more the corresponding upper normal limits. In some embodiments, the subject is at risk of adverse side effect from treatment with lanifibranor if the subject reference level of alanine transaminase is higher than at least 5.5 times, at least 6 times, at least 6.5 times, at least 7 times, at least 7.5 times, at least 8 times, at least 8.5 times, at least 9 times, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more the corresponding upper normal limit.

In some embodiments, the subject is at risk of adverse side effect from treatment with lanifibranor if the subject reference level of alkaline phosphatase is higher than at least 2 times or more the corresponding upper normal limit. In some embodiments, the subject is at risk of adverse side effect from treatment with lanifibranor if the subject reference level of alkaline phosphatase is higher than at least 2.5 times, at least 3 times, at least 3.5 times, at least 4 times, at least 4.5 times, at least 5 times, at least 5.5 times, at least 6 times, at least 6.5 times, at least 7 times, at least 7.5 times, at least 8 times, at least 8.5 times, at least 9 times, at least 9.5 times, at least 10 times or more the corresponding upper normal limit.

In some embodiments, the subject is at risk of adverse side effect from treatment with lanifibranor if the subject reference level of gamma-glutamyl transferase is higher than at least 2 times or more the corresponding upper normal limit. In some embodiments, the subject is at risk of adverse side effect from treatment with lanifibranor if the subject reference level of gamma-glutamyl transferase is higher than at least 2.5 times, at least 3 times, at least 3.5 times, at least 4 times, at least 4.5 times, at least 5 times, at least 5.5 times, at least 6 times, at least 6.5 times, at least 7 times, at least 7.5 times, at least 8 times, at least 8.5 times, at least 9 times, at least 9.5 times, at least 10 times or more the corresponding upper normal limit.

In some embodiments, the subject is at risk of adverse side effect from treatment with lanifibranor if the subject reference level of total bilirubin is higher than at least 1.05 times or more the corresponding upper normal limit. In some embodiments, the subject is at risk of adverse side effect from treatment with lanifibranor if the subject reference level of total bilirubin transferase is higher than at least 1.10 times, at least 1.15 times, at least 1.20 times, at least 1.25 times, at least 1.30 times, at least 1.35 times, at least 1.40 times, at least 1.45 times, at least 1.5 times, at least 1.55 times, at least 1.60 times, at least 1.65 times, at least 1.70 times, at least 1.75 times, at least 1.80 times, at least 1.85 times, at least 1.90 times, at least 1.95 times, at least 2 times, at least 2.5 times, at least 3.0 times, at least 3.5 times, at least 4 times, at least 5 times, at least 5.5 times, at least 6 times, at least 6.5 times, at least 7 times, at least 7.5 times, at least 8 times, at least 8.5 times, at least 9 times, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, or more the corresponding upper normal limit.

In some embodiments, the subject is at risk of adverse side effect from treatment with lanifibranor if the subject reference level of direct bilirubin is higher than at least 1.50 times or more the corresponding upper normal limit. In some embodiments, the subject is at risk of adverse side effect from treatment with lanifibranor if the subject reference level of direct bilirubin is higher than at least 1.55 times, at least 1.60 times, at least 1.65 times, at least 1.70 times, at least 1.75 times, at least 1.80 times, at least 1.85 times, at least 1.9 times, at least 1.95 times, at least 2 times, at least 2.1 times, at least 2.2 times, at least 2.3 times, at least 2.4 times, at least 2.5 times or more the corresponding upper normal limit.

In some embodiments, the subject is at risk of adverse side effect from treatment with lanifibranor if the subject reference level of PT/INR ratio is higher than at least 1.08 times or more the corresponding upper normal limit. In some embodiments, the subject is at risk of adverse side effect from treatment with lanifibranor if the subject reference level of PT/INR ratio is higher than at least 1.09 times, at least 1.10 times, at least 1.11 times, at least 1.12 times, at least 1.13 times, at least 1.14 times, at least 1.15 times, at least 1.16 times, at least 1.17 times, at least 1.18 times, at least 1.19 times, at least 1.20 times, at least 1.21 times, at least 1.22 times, at least 1.23 times, at least 1.24 times, at least 1.25 times, at least 1.30 times, at least 1.35 times, at least 1.40 times, at least 1.45 times, at least 1.50 times, at least 1.55 times, at least 1.60 times, at least 1.65 times, at least 1.70 times, at least 1.75 times, at least 1.80 times, at least 1.85 times, at least 1.90 times, at least 1.95 times, at least 2 times, at least 2.5 times, at least 3.0 times, at least 3.5 times, at least 4.0 times, at least 5 times, at least 5.5 times, at least 6 times, at least 6.5 times, at least 7 times, at least 7.5 times, at least 8 times, at least 8.5 times, at least 9 times, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, or more the corresponding upper normal limit.

In some embodiments, the subject is at risk of adverse side effect from treatment with lanifibranor if the panel of autoimmune disease biomarker is present and if the subject reference level of aspartate transaminase is higher than at least 5 times or more the corresponding upper normal limit.

In some embodiments, the subject is at risk of adverse side effect from treatment with lanifibranor if the panel of autoimmune disease biomarker is present and if the subject reference level of aspartate transaminase is higher than at least 10 times or more the corresponding upper normal limit.

In some embodiments, the subject is at risk of adverse side effect from treatment with lanifibranor if the panel of autoimmune disease biomarker is present and if the subject reference level of alanine transaminase is higher than at least 5 times or more the corresponding upper normal limit.

In some embodiments, the subject is at risk of adverse side effect from treatment with lanifibranor if the panel of autoimmune disease biomarker is present and if the subject reference level of alanine transaminase is higher than at least 10 times or more the corresponding upper normal limit.

In some embodiments, the subject is at risk of adverse side effect from treatment with lanifibranor if the panel of autoimmune disease biomarker is present and if the subject reference level of alkaline phosphatase is higher than at least 2 times or more the corresponding upper normal limit.

In some embodiments, the subject is at risk of adverse side effect from treatment with lanifibranor if the panel of autoimmune disease biomarker is present and if the subject reference level of gamma-glutamyl transferase is higher than at least 2 times or more the corresponding upper normal limit.

In some embodiments, the subject is at risk of adverse side effect from treatment with lanifibranor if the panel of autoimmune disease biomarker is present and if the subject reference level of total bilirubin is higher than at least 1.25 times or more the corresponding upper normal limit.

In some embodiments, the subject is at risk of adverse side effect from treatment with lanifibranor if the panel of autoimmune disease biomarker is present and if the subject reference level of direct bilirubin is higher than at least 1.51 times or more the corresponding upper normal limit.

In some embodiments, the subject is at risk of adverse side effect from treatment with lanifibranor if the panel of autoimmune disease biomarker is present and if the subject reference level of PT/INR ratio is higher than at least 1.08 times or more the corresponding upper normal limit.

In some embodiments, the subject is at risk of adverse side effect from treatment with lanifibranor if the panel of autoimmune disease biomarker is present and if the subject reference level of aspartate transaminase and the subject reference level of alanine transaminase are higher than at least 5 times or more the corresponding upper normal limit.

In some embodiments, the subject is at risk of adverse side effect from treatment with lanifibranor if:
the panel of autoimmune disease biomarker is present, and/or
the subject reference level of aspartate transaminase and/or the subject reference level of alanine transaminase are higher than at least 5 times or more the corresponding upper normal limit, and/or
the subject reference level of total bilirubin is higher than at least 1.25 times or more the corresponding upper normal limit.

In some embodiments, the subject is at risk of adverse side effect from treatment with lanifibranor if:
the panel of autoimmune disease biomarker is present, and/or
the subject reference level of aspartate transaminase and/or the subject reference level of alanine transaminase are higher than at least 3 times or more the corresponding upper normal limit, and/or
the subject reference level of total bilirubin is higher than at least 2 times or more the corresponding upper normal limit.

In some embodiments, the subject is at risk of adverse side effect from treatment with lanifibranor if:
the panel of autoimmune disease biomarker is present, and
the subject reference level of aspartate transaminase and/or the subject reference level of alanine transaminase are higher than at least 5 times or more the corresponding upper normal limit, and
the subject reference level of alkaline phosphatase is higher than at least 2 times or more the corresponding upper normal limit.

In some embodiments, the subject is at risk of adverse side effect from treatment with lanifibranor if:
the panel of autoimmune disease biomarker is present, and
the subject reference level of aspartate transaminase and/or the subject reference level of alanine transaminase are higher than at least 5 times or more the corresponding upper normal limit, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit.

In some embodiments, the subject is at risk of adverse side effect from treatment with lanifibranor if:
the panel of autoimmune disease biomarker is present, and
the subject reference level of aspartate transaminase and/or the subject reference level of alanine transaminase are higher than at least 5 times or more the corresponding upper normal limit, and/or
the subject reference level of alkaline phosphatase and the subject reference level of gamma-glutamyl transferase is higher than at least 2 times or more the corresponding upper normal limit, and/or
the subject reference level of total bilirubin is higher than at least 1.25 times or more the corresponding upper normal limit.

In some embodiments, the subject is at risk of adverse side effect from treatment with lanifibranor if:
the panel of autoimmune disease biomarker is present, and
the subject reference level of aspartate transaminase and/or the subject reference level of alanine transaminase are higher than at least 5 times or more the corresponding upper normal limit, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit, and/or
the subject reference level of total bilirubin is higher than at least 1.25 times or more the corresponding upper normal limit, and/or
the subject reference level of direct bilirubin is higher than at least 1.51 times or more the corresponding upper normal limit.

In some embodiments, the subject is at risk of adverse side effect from treatment with lanifibranor if:
the panel of autoimmune disease biomarker is present, and
the subject reference level of aspartate transaminase and/or the subject reference level of alanine transaminase are higher than at least 5 times or more the corresponding upper normal limit, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit, and/or
the subject reference level of total bilirubin is higher than at least 1.25 times or more the corresponding upper normal limit, and/or
the subject reference level of direct bilirubin is higher than at least 1.51 times or more the corresponding upper normal limit, and/or
the subject reference level of PT/INR ratio is higher than at least 1.08 times or more the corresponding upper normal limit.

In some embodiments, the subject is at risk of adverse side effect from treatment with lanifibranor if the subject reference level of aspartate transaminase and/or the subject reference level of alanine transaminase are higher than at least 5 times or more the corresponding upper normal limits.

In some embodiments, the subject is at risk of adverse side effect from treatment with lanifibranor if the subject reference level of aspartate transaminase and/or the subject reference level of alanine transaminase are higher than at least 10 times or more the corresponding upper normal limits.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of total bilirubin is higher than at least 1.25 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 3 times or more the corresponding upper normal limits, and/or
the subject reference level of total bilirubin is higher than at least 2 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of alkaline phosphatase is higher than at least 2 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit, and/or
the subject reference level of total bilirubin is higher than at least 1.25 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit, and/or
the subject reference level of total bilirubin is higher than at least 1.25 times or more the corresponding upper normal limit, and/or
the subject reference level of direct bilirubin is higher than at least 1.51 times or more the corresponding upper normal limit.

In some embodiments, the subject is at risk of adverse side effect from treatment with lanifibranor if:
the subject reference level of aspartate transaminase and/or the subject reference level of alanine transaminase are higher than at least 5 times or more the corresponding upper normal limits, and/or
the subject reference level of alkaline phosphatase is higher than at least 2 times or more the corresponding upper normal limit, and/or
the subject reference level of gamma-glutamyl transferase is higher than at least 2 times or more the corresponding upper normal limit, and/or
the subject reference level of total bilirubin is higher than at least 1.25 times or more the corresponding upper normal limit, and/or
the subject reference level of direct bilirubin is higher than at least 1.51 times or more the corresponding upper normal limit, and/or
the subject reference level of PT/INR ratio is higher than at least 1.08 times or more the corresponding upper normal limit.

In some embodiments, the subject is at risk of adverse side effect from treatment with lanifibranor:
if the panel of autoimmune disease biomarker is present, and/or
if the subject reference level of aspartate transaminase is higher than at least 10 times or more the corresponding upper normal limit; and/or
if the subject reference level of alanine transaminase is higher than at least 10 times or more the corresponding upper normal limit; and/or
if the subject reference level of aspartate transaminase is higher than at least 3 times or more the corresponding upper normal limit and the subject reference level of total bilirubin is higher than at least 2 times or more the corresponding upper normal limit; and/or
if the subject reference level of alanine transaminase is higher than at least 3 times or more the corresponding upper normal limit and the subject reference level of total bilirubin is higher than at least 2 times or more the corresponding upper normal limit; and/or
if the subject reference level of alkaline phosphatase is higher than at least 2 times or more the corresponding upper normal limit, the subject reference level of gamma-glutamyl transferase is higher than at least 2 times or more the corresponding upper normal limit, the subject reference level of total bilirubin is higher than at least 1.25 times or more the corresponding upper normal limit, the subject reference level of direct bilirubin is higher than at least 1.51 times or more the corresponding upper normal limit, and the subject reference level of PT/INR ratio is higher than at least 1.08 times or more the corresponding upper normal limit.

In some embodiments, the subject is at risk of adverse side effect from treatment with lanifibranor:
if the panel of autoimmune disease biomarker is present, and/or
if the subject reference level of aspartate transaminase is higher than at least 5 times or more the corresponding upper normal limit, and/or
if the subject reference level of alanine transaminase is higher than at least 5 times or more the corresponding upper normal limit, and/or
if the subject reference level of alkaline phosphatase is higher than at least 2 times or more the corresponding upper normal limit, and
if the subject reference level of gamma-glutamyl transferase is higher than at least 2 times or more the corresponding upper normal limit, and
if the subject reference level of total bilirubin is higher than at least 1.25 times or more the corresponding upper normal limit, and
if the subject reference level of direct bilirubin is higher than at least 1.51 times or more the corresponding upper normal limit, and
if the subject reference level of PT/INR ratio is higher than at least 1.08 times or more the corresponding upper normal limit.

In some embodiments, a subject is selected for treatment with lanifibranor:
if the panel of autoimmune disease biomarker is present, wherein the panel of autoimmune biomarkers comprises the combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and/or
if the subject reference level of aspartate transaminase is higher than at least 5 times or more the corresponding upper normal limit, advantageously if the subject reference level of aspartate transaminase is higher than at least 160 U/L for women and at least 200 U/L for men, and/or
if the subject reference level of alanine transaminase is higher than at least 5 times or more the corresponding upper normal limit, advantageously if the subject reference level of alanine transaminase is higher than at least 165 U/L for women and at least 205 U/L for men, and/or
if the subject reference level of alkaline phosphatase is higher than at least 2 times or more the corresponding upper normal limit, advantageously if the subject reference level of alkaline phosphatase is higher than at least 208 U/L for women and at least 258 U/L for men, and/or
if the subject reference level of gamma-glutamyl transferase is higher than at least 2 times or more the corresponding upper normal limit, advantageously if the subject reference level of gamma-glutamyl transferase is higher than at least 72 U/L for women and at least 122 U/L for men, and/or
if the subject reference level of total bilirubin is higher than at least 1.25 times or more the corresponding upper normal limit, advantageously if the subject reference level of total bilirubin is higher than at least 25.6 μmol/L, and/or
if the subject reference level of direct bilirubin is higher than at least 1.51 times or more the corresponding upper normal limit, advantageously if the subject reference level of direct bilirubin is higher than at least 7.7 μmol/L, and/or
if the subject reference level of PT/INR ratio is higher than at least 1.08 times or more the corresponding upper normal limit, advantageously if the subject reference level of PT/INR ratio is higher than at least 1.30.

In another aspect, the present disclosure relates to a method for reducing the risk or occurrence of adverse effect in a subject suffering from liver disease, the method comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) administering to said subject an effective amount of lanifibranor if:
   (i) the panel of autoimmune disease biomarkers is absent in the reference sample, and
   (ii) the subject reference level of at least one liver biomarker is below than the reference level of the at least one liver biomarker.

According to the present disclosure, steps a), b) and c) are performed as defined above. According to the present disclosure, the panel of autoimmune disease biomarkers and the at least one liver biomarkers are as defined above.

In some embodiments, the method for reducing the risk or occurrence of adverse effect in a subject suffering from liver disease, the method comprises:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) administering to said subject an effective amount of lanifibranor if:
   (i) the panel of autoimmune disease biomarkers is absent in the reference sample, and
   (ii) the subject reference level of at least one liver biomarker is below than the reference level of the at least one liver biomarker,
   wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof, and
   wherein the panel of autoimmune disease biomarkers is a combination of at least two autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the method for reducing the risk or occurrence of adverse effect in a subject suffering from liver disease, the method comprises:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) administering to said subject an effective amount of lanifibranor if:
   (i) the panel of autoimmune disease biomarkers is absent in the reference sample, and
   (ii) the subject reference level of at least one liver biomarker is below than the reference level of the at least one liver biomarker,
   wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof, and
   wherein the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In another aspect, the present disclosure relates to a method for reducing the risk or occurrence of adverse effect caused by lanifibranor treatment in a subject suffering from liver disease, the method comprising:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) wherein the risk or occurrence of adverse effect caused by lanifibranor treatment in a subject suffering from liver disease is reduced when:
   (i) the panel of autoimmune disease biomarkers is absent in the reference sample, and
   (ii) the subject reference level of at least one liver biomarker is below than the reference level of the at least one liver biomarker.

According to the present disclosure, steps a), b) and c) are performed as defined above. According to the present disclosure, the panel of autoimmune disease biomarkers and the at least one liver biomarkers are as defined above.

In some embodiments, the method for reducing the risk or occurrence of adverse effect caused by lanifibranor treatment in a subject suffering from liver disease comprises:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) wherein the risk or occurrence of adverse effect caused by lanifibranor treatment in a subject suffering from liver disease is reduced when:
   (i) the panel of autoimmune disease biomarkers is absent in the reference sample, and
   (ii) the subject reference level of at least one liver biomarker is below than the reference level of the at least one liver biomarker,
   wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof, and
   wherein the panel of autoimmune disease biomarkers is a combination of at least two autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the method for reducing the risk or occurrence of adverse effect caused by lanifibranor treatment in a subject suffering from liver disease comprises:
a) determining the presence or the absence of a panel of autoimmune disease biomarkers in a reference sample obtained from the subject,
b) determining the subject reference level of at least one liver biomarker in a reference sample obtained from the subject,
c) comparing the subject reference level of the at least one liver biomarker to the reference level of the at least one liver biomarker,
d) wherein the risk or occurrence of adverse effect caused by lanifibranor treatment in a subject suffering from liver disease is reduced when:
   (i) the panel of autoimmune disease biomarkers is absent in the reference sample, and
   (ii) the subject reference level of at least one liver biomarker is below than the reference level of the at least one liver biomarker,
   wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof, and
   wherein the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

In some embodiments, the method for reducing the risk or occurrence of adverse effect caused by lanifibranor treatment in a subject suffering from liver disease comprises:
a) determining the presence or the absence of a panel of autoimmune disease biomarkers in a reference sample obtained from the subject,
b) determining the subject reference level of at least one liver biomarker in a reference sample obtained from the subject, wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof;
c) comparing the subject reference level of the at least one liver biomarker to the reference level of the at least one liver biomarker,
d) wherein the risk or occurrence of adverse effect caused by lanifibranor treatment in a subject suffering from liver disease is reduced when:
   (i) the panel of autoimmune disease biomarkers is absent in the reference sample, wherein the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody, and
   (ii) at least one of the following conditions is satisfied:
      the subject reference level of aspartate transaminase is equal or below than the reference level of aspartate transaminase,
      the subject reference level of alanine transaminase is equal or below than the reference level of alanine transaminase,
      the subject reference level of alkaline phosphatase is equal or below than the reference level of alkaline phosphatase,
      the subject reference level of gamma-glutamyl transferase is equal or below than the reference level of gamma-glutamyl transferase,
      the subject reference level of total bilirubin is equal or below than the reference level of total bilirubin,
      the subject reference level of direct bilirubin is equal or below than the reference level of direct bilirubin, and/or
      the subject reference level of PT/INR ratio is equal or below than the reference level of PT/INR ratio.

In another aspect, the present disclosure relates to a method for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of at least one liver biomarker in a baseline sample obtained from the subject; and
c) comparing the subject baseline level of the at least one liver biomarker to the corresponding upper normal limit of the at least one liver biomarker, to the reference level of the at least one liver biomarker and to the subject reference level of the at least one liver biomarker;
   wherein subject's risk of experiencing the adverse side effect when:
(i) the panel of autoimmune disease biomarkers is present in a baseline sample obtained from the subject, and/or
(ii) the subject baseline level of the at least one liver biomarker is higher than the reference level of said at least one liver biomarker; or
(iii) the subject baseline level of the at least one liver biomarker is higher than 300 U/L; or
(iv) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
(v) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L.

According to the present disclosure, steps a), b) and c) are performed as defined above.

In some embodiments, the subject is at risk of experiencing an adverse side effect when the panel of autoimmune disease biomarkers is present in a baseline sample obtained from the subject. According to the present disclosure, the panel of autoimmune disease biomarkers is as defined above.

In some embodiments, the subject is at risk of experiencing an adverse side effect when the subject baseline level of the at least one liver biomarker is at least 1.5 times, at least 2 times, at least 2.5 times, at least 3 times, at least 3.5 times, at least 4 times, at least 4.5 times, at least 5 times, at least 5.5 times higher, at least 6 times at least 6.5 times, at least 7 times, at least 7.5 times, at least 8 times, at least 8.5 times, at least 9 times higher, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more higher than the corresponding upper normal limit of the at least one liver biomarker or than the subject reference level of the at least one liver biomarker in said baseline sample. According to the present disclosure, the at least one liver biomarker is as defined above. In some embodiments, the subject is at risk of experiencing an adverse side effect when the subject baseline level of the at least one liver biomarker is at least 1.5 times, at least 2 times, at least 2.5 times, at least 3 times, at least 3.5 times, at least 4 times, at least 4.5 times, at least 5 times, at least 5.5 times higher, at least 6 times at least 6.5 times, at least 7 times, at least 7.5 times, at least 8 times, at least 8.5 times, at least 9 times higher, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more higher than the corresponding upper normal limit of the at least one liver biomarker or than the subject reference level of the at least one liver biomarker in said baseline sample, if the subject baseline level of the at least one liver biomarker remains at least at the same level during specific period of time. In some embodiments, the specific period of time is at least an hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, at least 96 hours. In some embodiments, the specific period of time is between at least an hour and at least 96 hours, for example between at least 24 hours and at least 96 hours, for example between at least 48 hours and at least 96 hours, for example between at least 48 hours and at least 72 hours.

In some embodiments, the subject is at risk of experiencing an adverse side effect when the subject baseline level of the at least one liver biomarker is higher than the reference level of said at least one liver biomarker.

In some embodiments, the subject is at risk of experiencing an adverse side effect when the subject baseline level of aspartate transaminase is at least 1.5 times, at least 2 times, at least 2.5 times, at least 3 times, at least 3.5 times, at least 4 times, at least 4.5 times, at least 5 times, at least 5.5 times higher, at least 6 times at least 6.5 times, at least 7 times, at least 7.5 times, at least 8 times, at least 8.5 times, at least 9 times higher, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more higher than the corresponding upper normal limit of aspartate transaminase or than the subject reference level of aspartame transaminase in said baseline sample. In some embodiments, the subject is at risk of experiencing an adverse side effect when the subject baseline level of aspartate transaminase is at least 8 times higher than the corresponding upper normal limit of aspartate transaminase or than the subject reference level of aspartame transaminase in said baseline sample. In some embodiments, the subject is at risk of experiencing an adverse side effect when the subject baseline level of aspartate transaminase is at least 10 times higher than the corresponding upper normal limit of aspartate transaminase or than the subject reference level of aspartame transaminase in said baseline sample.

In some embodiments, the subject is at risk of experiencing an adverse side effect when the subject baseline level of aspartate transaminase is at least 5 times than the corresponding upper normal limit of aspartate transaminase or than the subject reference level of aspartame transaminase in said baseline sample. In some embodiments, the subject is at risk of experiencing an adverse side effect when the subject baseline level of aspartate transaminase is higher than the reference level of aspartate transaminase.

In some embodiments, the subject is at risk of experiencing an adverse side effect when the subject baseline level of alanine transaminase is at least 1.5 times, at least 2 times, at least 2.5 times, at least 3 times, at least 3.5 times, at least 4 times, at least 4.5 times, at least 5 times, at least 5.5 times higher, at least 6 times at least 6.5 times, at least 7 times, at least 7.5 times, at least 8 times, at least 8.5 times, at least 9 times higher, at least 9.5 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 21 times, at least 42 times, at least 43 times, at least 44 times, at least 45 times, at least 46 times, at least 47 times, at least 48 times, at least 49 times, at least 50 times, at least 51 times, at least 52 times, at least 53 times, at least 54 times, at least 55 times, at least 56 times, at least 57 times, at least 58 times, at least 59 times, at least 60 times, at least 61 times, at least 62 times, at least 63 times, at least 64 times, at least 65 times, at least 66 times, at least 67 times, at least 68 times, at least 69 times, at least 70 times, or more higher than the corresponding upper normal limit of aspartate transaminase or than the subject reference level of aspartame transaminase in said baseline sample. In some embodiments, the subject is at risk of experiencing an adverse side effect when the subject baseline level of alanine transaminase is at least 8 times higher than the corresponding upper normal limit of aspartate transaminase or than the subject reference level of aspartame transaminase in said baseline sample. In some embodiments, the subject is at risk of experiencing an adverse side effect when the subject baseline level of alanine transaminase is at least 10 times higher than the corresponding upper normal limit of alanine transaminase or than the subject reference level of aspartame transaminase in said baseline sample. In some embodiments, the subject is at risk of experiencing an adverse side effect when the subject baseline level of alanine transaminase is at least 5 times higher than the corresponding upper normal limit of alanine transaminase or than the subject reference level of aspartame transaminase in said baseline sample. In some embodiments, the subject is at risk of experiencing an adverse side effect when the subject baseline level of aspartate transaminase is higher than the reference level of alanine transaminasc.

In some embodiments, the subject is at risk of experiencing an adverse side effect when the subject baseline level of the at least one liver biomarker is higher than 300 IU/L, in particular higher than 310 IU/L, in particular higher than 320 IU/L, in particular higher than 330 IU/L, in particular higher than 340 IU/L, in particular higher than 350 IU/L, in particular higher than 360 IU/L, in particular higher than 370 IU/L, in particular higher than 380 IU/L, in particular higher than 390 IU/L, in particular higher than 400 IU/L, in particular higher than 410 IU/L, in particular higher than 420 IU/L, in particular higher than 430 IU/L, in particular higher than 440 IU/L, in particular higher than 450 IU/L, in particular higher than 460 IU/L, in particular higher than 470 IU/L, in particular higher than 480 IU/L, in particular higher than 490 IU/L, in particular higher than 500 IU/L, in particular higher than 510 IU/L, in particular higher than 520 IU/L, in particular higher than 530 IU/L, in particular higher than 540 IU/L, in particular higher than 550 IU/L, in particular higher than 560 IU/L, in particular higher than 570 IU/L, in particular higher than 580 IU/L, in particular higher than 590 IU/L, in particular higher than 600 IU/L, in particular higher than 610 IU/L, in particular higher than 620 IU/L, in particular higher than 630 IU/L, in particular higher than 640 IU/L, in particular higher than 650 IU/L, in particular higher than 660 IU/L, in particular higher than 670 IU/L, in particular higher than 680 IU/L, in particular higher than 690 IU/L, in particular higher than 700 IU/L, or more higher. In another aspect, the present disclosure relates to a method for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of at least one liver biomarker in a baseline sample obtained from the subject; and
c) comparing the subject baseline level of the first liver biomarker to the upper normal limit of the first liver biomarker, to reference level of the first liver biomarker and to the subject reference level of the first liver biomarker; and
d) comparing the subject baseline level of the second liver biomarker to the upper normal limit of the second liver biomarker, to the reference level of the second liver biomarker and to the subject reference level of the second liver biomarker;
   wherein the subject is at risk of experiencing an adverse side effect when:
(i) the panel of autoimmune disease biomarkers is present in a baseline sample obtained from the subject, and/or
(ii) the subject baseline level of the first liver biomarker is 10 times higher than the upper normal limit of the first liver biomarker, or is 2 times higher than reference level of the first liver biomarker, or is higher than 300 IU/L; or
(iii) the subject baseline level of the second liver biomarker is 10 times higher than the upper normal limit of the second liver biomarker, or is 2 times higher than reference level of the second liver biomarker, or is higher than 300 IU/L; or
(iv) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
(v) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased.

In some embodiments, the first liver biomarker is aspartate transaminase and the second liver biomarker is alanine transaminase. In some embodiments, the at least other liver biomarkers are total bilirubin or INR ratio. In some embodiments, the subject baseline level of at least one other liver biomarkers has increased, when the subject baseline level of at least one other liver biomarkers is at least 1 time higher than the upper normal limit of said at least one other liver biomarkers, in particular at least 1.5 times, in particular at least 2 times, in particular at least 2.5 times, in particular at least 3 times, in particular at least 3.5 times, in particular at least 4 times, in particular at least 4.5 times, in particular at least 5 times, in particular at least 5.5 times, in particular at least 6 times, in particular at least 6.5 times, in particular at least 7 times, in particular at least 7.5 times, in particular at least 8 times, in particular at least 8.5 times, in particular at least 9 times, in particular at least 9.5 times, in particular at least 10 times, in particular at least 11 times, in particular at least 12 times, in particular at least 13 times, in particular at least 14 times, in particular at least 15 times, in particular at least 16 times, in particular at least 17 times, in particular at least 18 times, in particular at least 19 times, in particular at least 20 times, in particular at least 21 times, in particular at least 22 times, in particular at least 23 times, in particular at least 24 times, in particular at least 25 times, in particular at least 26 times, in particular at least 27 times, in particular at least 28 times, in particular at least 29 times, in particular at least 30 times, in particular at least 31 times, in particular at least 32 times, in particular at least 33 times, in particular at least 34 times, in particular at least 35 times, in particular at least 36 times, in particular at least 37 times, in particular at least 38 times, in particular at least 39 times, in particular at least 40 times, in particular at least 21 times, in particular at least 42 times, in particular at least 43 times, in particular at least 44 times, in particular at least 45 times, in particular at least 46 times, in particular at least 47 times, in particular at least 48 times, in particular at least 49 times, in particular at least 50 times, in particular at least 51 times, in particular at least 52 times, in particular at least 53 times, in particular at least 54 times, in particular at least 55 times, in particular at least 56 times, in particular at least 57 times, in particular at least 58 times, in particular at least 59 times, in particular at least 60 times, in particular at least 61 times, in particular at least 62 times, in particular at least 63 times, in particular at least 64 times, in particular at least 65 times, in particular at least 66 times, in particular at least 67 times, in particular at least 68 times, in particular at least 69 times, in particular at least 70 times higher than the upper normal limit of said at least one other liver biomarkers. In some embodiments, the at least other liver biomarkers are total bilirubin.

In some embodiments, the subject has a risk of experiencing an adverse side effect when:
(i) the subject baseline level of aspartate transaminase is at least 3 times higher than the upper normal limit of aspartate transaminase or the subject baseline level of aspartate transaminase is at least 3 times higher than the subject reference level of aspartate transaminase and the subject baseline level of total bilirubin has increased; or
(ii) the subject baseline level of alanine transaminase is at least 3 times higher than the upper normal limit of alanine transaminase or the subject baseline level of alanine transaminase is at least 3 times higher than the subject reference level of alanine transaminase and the subject baseline level of total bilirubin has increased.

In some embodiments, the subject has a risk of experiencing an adverse side effect when:
(i) the subject baseline level of aspartate transaminase is at least 3 times higher than the upper normal limit of aspartate transaminase or the subject baseline level of aspartate transaminase is at least 3 times higher than the subject reference level of aspartate transaminase and the subject baseline level of total bilirubin is at least 1.5 times, in particular at least 2 times, in particular at least 2.5 times, in particular at least 3 times, in particular at least 3.5 times, in particular at least 4 times, in particular at least 4.5 times, in particular at least 5 times, in particular at least 5.5 times, in particular at least 6 times, in particular at least 6.5 times, in particular at least 7 times, in particular at least 7.5 times, in particular at least 8 times, in particular at least 8.5 times, in particular at least 9 times, in particular at least 9.5 times, in particular at least 10 times higher than the upper normal limit of total bilirubin; or
(ii) the subject baseline level of alanine transaminase is at least 3 times higher than the upper normal limit of alanine transaminase or the subject baseline level of alanine transaminase is at least 3 times higher than the subject reference level of alanine transaminase and the subject baseline level of total bilirubin is at least 1.5 times, in particular at least 2 times, in particular at least 2.5 times, in particular at least 3 times, in particular at least 3.5 times, in particular at least 4 times, in particular at least 4.5 times, in particular at least 5 times, in particular at least 5.5 times, in particular at least 6 times, in particular at least 6.5 times, in particular at least 7 times, in particular at least 7.5 times, in particular at least 8 times, in particular at least 8.5 times, in particular at least 9 times, in particular at least 9.5 times, in particular at least 10 times higher than the upper normal limit of total bilirubin.

In some embodiments, the subject has a risk of experiencing an adverse side effect when:
(i) the subject baseline level of aspartate transaminase is at least 3 times higher than the upper normal limit of aspartate transaminase or the subject baseline level of aspartate transaminase is at least 3 times higher than the subject reference level of aspartate transaminase and the subject baseline level of total bilirubin is at least 2 times higher than the upper normal limit of total bilirubin; or
(ii) the subject baseline level of alanine transaminase is at least 3 times higher than the upper normal limit of alanine transaminase or the subject baseline level of alanine transaminase is at least 3 times higher than the subject reference level of alanine transaminase and the subject baseline level of total bilirubin is at least 2 times higher than the upper normal limit of total bilirubin.

In some embodiments, the subject is at risk of experiencing an adverse side effect when the subject baseline level of the at least one liver biomarker is higher than 300 IU/L, in particular higher than 310 IU/L, in particular higher than 320 IU/L, in particular higher than 330 IU/L, in particular higher than 340 IU/L, in particular higher than 350 IU/L, in particular higher than 360 IU/L, in particular higher than 370 IU/L, in particular higher than 380 IU/L, in particular higher than 390 IU/L, in particular higher than 400 IU/L, in particular higher than 410 IU/L, in particular higher than 420 IU/L, in particular higher than 430 IU/L, in particular higher than 440 IU/L, in particular higher than 450 IU/L, in particular higher than 460 IU/L, in particular higher than 470 IU/L, in particular higher than 480 IU/L, in particular higher than 490 IU/L, in particular higher than 500 IU/L, in particular higher than 510 IU/L, in particular higher than 520 IU/L, in particular higher than 530 IU/L, in particular higher than 540 IU/L, in particular higher than 550 IU/L, in particular higher than 560 IU/L, in particular higher than 570 IU/L, in particular higher than 580 IU/L, in particular higher than 590 IU/L, in particular higher than 600 IU/L, in particular higher than 610 IU/L, in particular higher than 620 IU/L, in particular higher than 630 IU/L, in particular higher than 640 IU/L, in particular higher than 650 IU/L, in particular higher than 660 IU/L, in particular higher than 670 IU/L, in particular higher than 680 IU/L, in particular higher than 690 IU/L, in particular higher than 700 IU/L, or more higher. In some embodiments, the subject is at risk of experiencing an adverse side effect when the subject baseline level of aspartate transaminase is at least 10 times higher than the corresponding upper normal limit of alanine transaminase or than the subject reference level of aspartame transaminase in said baseline sample. In some embodiments, the subject is at risk of experiencing an adverse side effect when the subject baseline level of aspartate transaminase is higher than 500 U/L.

In some embodiments, the subject is at risk of experiencing an adverse side effect when the subject baseline level of alanine transaminase is at least 10 times higher than the corresponding upper normal limit of alanine transaminase or than the subject reference level of aspartame transaminase in said baseline sample. In some embodiments, the subject is at risk of experiencing an adverse side effect when the subject baseline level of alanine transaminase is higher than 500 U/L.

In some embodiments, the determining of the presence of a panel of autoimmune disease biomarkers and/or the determining of the subject baseline level of at least one liver biomarker in a baseline sample obtained from the subject, are made at regular interval of time for a determined duration. In some embodiments, the regular interval of time is at least every 1 hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, at least every 1 month, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 1 year, at least every 2 years, at least every 3 years, at least every 4 years, at least every 5 years, at least every 6 years, at least every 7 years, at least every 8 years, at least every 9 years, at least every 10 years, at least every 11 years, at least every 12 years.

In some embodiments, the determined duration is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more. The determined duration, and a fortiori the end of the treatment, can vary depending upon the physical condition of the subject to be treated, a professional assessment of the medical situation, and other relevant factors.

In some embodiments, the determining of the subject baseline level of at least one liver biomarker in a baseline sample obtained from the subject is made at regular interval of time of at least every 1 hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, at least every 1 month, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 1 year, at least every 2 years, at least every 3 years, at least every 4 years, at least every 5 years, at least every 6 years, at least every 7 years, at least every 8 years, at least every 9 years, at least every 10 years, at least every 11 years, at least every 12 years for a determined duration of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more.

In some embodiments, the determining of the subject baseline level of at least one liver biomarker in a baseline sample obtained from the subject is made:
at regular interval of time of at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 16 days, at least every 17 days, at least every 18 days, at least every 19 days, at least every 20 days, at least every 21 days, at least every 22 days, at least every 23 days, at least every 24 days, at least every 25 days, at least every 26 days, at least every 27 days, at least every 28 days, at least every 29 days, at least every 30 days, at least every 31 days, at least every 1 week, at least every 2 weeks, at least every 3 weeks, at least every 4 weeks, at least every 5 weeks, at least every 6 weeks, for a determined duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years, and then
continued at regular interval of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more.

In some embodiments, the determining of the subject baseline level of at least one liver biomarker in a baseline sample obtained from the subject is made at regular interval of time of 6 weeks for a determined duration of 10 months, and is then continued at regular interval of 3 months until the end of lanifibranor treatment.

In some embodiments, the determining of the subject baseline level of at least one liver biomarker in a baseline sample obtained from the subject is made at regular interval of time of 4 weeks for a determined duration of 10 months, and is then continued at regular interval of 3 months until the end of lanifibranor treatment.

In some embodiments, the determining of the subject baseline level of at least one liver biomarker in a baseline sample obtained from the subject is made at regular interval of time of 4 weeks for a determined duration of 10 months, and is then continued at regular interval of 6 weeks until the end of lanifibranor treatment.

In some embodiments, the determining of the subject baseline level of at least one liver biomarker in a baseline sample obtained from the subject is made at regular interval of time of 4 weeks for a determined duration of 48 weeks, and is then continued at regular interval of 6 weeks until the end of lanifibranor treatment.

In another aspect of the disclosure, the present disclosure relates to a method for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of a first liver biomarker and the subject baseline level of a second liver biomarker in a baseline sample obtained from the subject; and
c) comparing the subject baseline level of the first liver biomarker to the upper normal limit of the first liver biomarker, to reference level of the first liver biomarker and to the subject reference level of the first liver biomarker; and
d) comparing the subject baseline level of the second liver biomarker to the upper normal limit of the second liver biomarker, to the reference level of the second liver biomarker and to the subject reference level of the second liver biomarker;
e) perform close monitoring for a specific period of time if:
   a. the subject reference level of the first liver biomarker is below than 1.5 times the upper normal limit of the first liver biomarker, or
   b. the subject reference level of the second liver biomarker is below than 1.5 times the upper normal limit of the second liver biomarker,
   and
   a. the subject baseline level of the first liver biomarker is equal or higher than 3 times the upper normal limit of the first liver biomarker but below than 5 times the upper normal limit of the first liver biomarker, or
   b. the subject baseline level of the second liver biomarker is equal or higher than 3 times the upper normal limit of the second liver biomarker but below than 5 times the upper normal limit of the second liver biomarker; or
   c. the subject baseline level of the first liver biomarker is equal or higher than 5 times the upper normal limit of the first liver biomarker, or
   d. the subject baseline level of the second liver biomarker is equal or higher than 5 times the upper normal limit of the second liver biomarker;
      wherein the subject is at risk of experiencing the adverse side effect when:
(i) the panel of autoimmune disease biomarkers is present in a baseline sample obtained from the subject, or
(ii) the subject baseline level of the first liver biomarker increases or remains to at least the reference level of the first liver biomarker, or
(iii) the subject baseline level of the second liver biomarker increases or remains to at least the reference level of the second liver biomarker, or
(iv) the subject baseline level of the first liver biomarker is at least 5 times higher than the upper normal limit of the first liver biomarker, or
(v) the subject baseline level of the second liver biomarker is at least 5 times higher than the upper normal limit of the second liver biomarker, or
(vi) the subject baseline level of the first liver biomarker increases or remains to at least 3 times the upper normal limit of the first liver biomarker and the subject baseline level of two others liver biomarkers has increased, or
(vii) the subject baseline level of the second liver biomarker increases or remains to at least 3 times the upper normal limit of the second liver biomarker and the subject baseline level of two others liver biomarkers has increased, or
(viii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
(ix) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia.

In some embodiments, the first liver biomarker is aspartate transaminase and the second liver biomarker is alanine transaminase. In some embodiments, the two others liver biomarkers are total bilirubin and INR ratio.

In some embodiments, the subject is at risk of experiencing an adverse side effect when:
the subject baseline level of aspartate transaminase is at least 3 times higher than the upper normal limit of aspartate transaminase or the subject baseline level of alanine transaminase is at least 3 times higher than the upper normal limit of alanine transaminase, and
the subject baseline level of total bilirubin is higher than the reference level of total bilirubin or the subject baseline level of PT/INR ratio is higher than the reference level of PT/INR ratio.

In some embodiments, the subject is at risk of experiencing an adverse side effect when:
the subject baseline level of aspartate transaminase is at least 3 times higher than the upper normal limit of aspartate transaminase or the subject baseline level of alanine transaminase is at least 3 times higher than the upper normal limit of alanine transaminase, and
the subject baseline level of total bilirubin equal or higher than at least 2 times the upper normal limit of total bilirubin, or
the subject baseline level of PT/INR ratio is higher than 1.5.

In some embodiments, the subject is at risk of experiencing an adverse side effect when:
the subject baseline level of aspartate transaminase is at least 2 times higher than the subject reference level of aspartame transaminase in said baseline sample or the subject baseline level of alanine transaminase is at least 2 times higher than the subject reference level of alanine transaminase in said baseline sample, and
the subject baseline level of total bilirubin is higher than the reference level of total bilirubin or the subject baseline level of PT/INR ratio is higher than the reference level of PT/INR ratio.

In some embodiments, the subject is at risk of experiencing an adverse side effect when:
the subject baseline level of aspartate transaminase is at least 2 times higher than the subject reference level of aspartame transaminase in said baseline sample or the subject baseline level of alanine transaminase is at least 2 times higher than the subject reference level of alanine transaminase in said baseline sample, and
the subject baseline level of total bilirubin equal or higher than at least 2 times the upper normal limit of total bilirubin, or
the subject baseline level of PT/INR ratio is higher than 1.5.

In some embodiments, the subject is at risk of experiencing an adverse side effect when:
the subject baseline level of aspartate transaminase is at least 3 times higher than the subject reference level of aspartame transaminase in said baseline sample, and
the subject baseline level of total bilirubin equal or higher than at least 2 times the upper normal limit of total bilirubin.

In some embodiments, the subject is at risk of experiencing an adverse side effect when:
the subject baseline level of alanine transaminase is at least 3 times higher than the subject reference level of alanine transaminase in said baseline sample, and
the subject baseline level of total bilirubin equal or higher than at least 2 times the upper normal limit of total bilirubin.

In some embodiments, the subject is at risk of experiencing an adverse side effect when:
the subject baseline level of aspartate transaminase is at least 3 times higher than the subject reference level of aspartame transaminase in said baseline sample, and
the subject baseline level of alanine transaminase is at least 3 times higher than the subject reference level of alanine transaminase in said baseline sample, and
the subject baseline level of total bilirubin equal or higher than at least 2 times the upper normal limit of total bilirubin.

In case the subject is at risk of experiencing an adverse side effect, the treatment with lanifibranor should be discontinued. In some embodiments, if the subject is not at risk of experiencing a side effect, the method further comprises administering lanifibranor to the subject.

In some embodiments, the determining of the presence of a panel of autoimmune disease biomarkers and the determining of the subject baseline level of at least one liver biomarker in a baseline sample obtained from the subject, are made at regular interval of time. In some embodiments, the interval of time is at least every hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every 36 hours, at least every 48 hours, at least every 60 hours, at least every 72 hours, at least every 96 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, at least every 1 month, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 1 year, at least every 2 years, at least every 3 years, at least every 4 years, at least every 5 years, at least every 6 years, at least every 7 years, at least every 8 years, at least every 9 years, at least every 10 years, at least every 11 years, at least every 12 years.

In some embodiments, the specific period of time is of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, at least 96 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks. In some embodiments, the specific period of time is between at least 1 hour and 1 week, for example between at least 1 hour and at least 96 hours, for example between at least 24 hours and at least 96 hours, for example between at least 48 hours and at least 96 hours, for example between at least 48 hours and at least 72 hours.

In some embodiments, the method for monitoring a subject's risk of experiencing an adverse side effect caused by lanifibranor treatment, comprises:
a) determining the presence of a panel of autoimmune disease biomarkers in a baseline sample obtained from the subject,
b) determining the subject baseline level of aspartate transaminase and the subject baseline level of alanine transaminase in a baseline sample obtained from the subject; and
c) comparing the subject baseline level of aspartate transaminase to the upper normal limit of aspartate transaminase, to the reference level of aspartate transaminase and to the subject reference level of aspartate transaminase; and
d) comparing the subject baseline level of alanine transaminase to the upper normal limit of alanine transaminase, to the reference level of alanine transaminase and to the subject reference level of alanine transaminase; and
e) perform close monitoring for a specific period of time if:
   a. the subject reference level of aspartate transaminase is below than 1.5 times the upper normal limit of aspartate transaminase, or
   b. the subject reference level of alanine transaminase is below than 1.5 times the upper normal limit of alanine transaminase,
   and
   a. the subject baseline level of aspartate transaminase is equal or higher than 3 times the upper normal limit of aspartate transaminase, but below 5 times the upper normal limit of the aspartate transaminase or alanine transaminase, or
   b. the subject baseline level of alanine transaminase is equal or higher than 3 times the upper normal limit of alanine transaminase, but below 5 times the upper normal limit of alanine transaminase, or
   c. the subject baseline level of aspartate transaminase is equal or higher than 5 times the upper normal limit of aspartate transaminase, or
   d. the subject baseline level of alanine transaminase is equal or higher than 5 times the upper normal limit of alanine transaminase;
      wherein the subject is at risk of experiencing the adverse side effect when:
(i) the panel of autoimmune disease biomarkers is present in a baseline sample obtained from the subject, or
(ii) the subject baseline level of aspartate transaminase increases or remains to at least the reference level of aspartate transaminase, or
(iii) the subject baseline level of alanine transaminase increases or remains to at least the reference level of alanine transaminase, or
(iv) the subject baseline level of aspartate transaminase is at least 5 times higher than the upper normal limit of aspartate transaminase, or
(v) the subject baseline level of alanine transaminase is at least 5 times higher than the upper normal limit of alanine transaminase, or
(vi) the subject baseline level of aspartate transaminase increases or remains to at least 3 times the upper normal limit of aspartate transaminase and the level of total bilirubin and PT/INR ratio has increased, or
(vii) the subject baseline level of alanine transaminase increases or remains to at least 3 times the upper normal limit of alanine transaminase and the level of total bilirubin and PT/INR ratio has increased, or
(viii) the subject baseline level of aspartate transaminase increases or remains to at least 3 times the upper normal limit of aspartate transaminase, with the appearance of fatigue, nausea, vomiting, right upper quadrant pain or tenderness, fever, rash, and/or eosinophilia, or
(ix) the subject baseline level of alanine transaminase increases or remains to at least 3 times the upper normal limit of alanine transaminase, with the appearance of fatigue, nausea, vomiting, right upper quadrant pain or tenderness, fever, rash, and/or eosinophilia.

According to the above-mentioned embodiments, the first liver biomarker is aspartate transaminase, the second liver biomarker is alanine transaminase and the two others liver biomarkers are total bilirubin and PT/INR ratio. Advantageously, the level of total bilirubin has increased and is at least 2 times higher than the upper normal limit of the total bilirubin. Advantageously, the level of total bilirubin has increased and is at least 2 times higher than the upper normal limit of the total bilirubin. Advantageously, the PT/INR ratio has increased and is at least 1.08 times higher than the upper normal limit of the PT/INR ratio. Advantageously, the level of total bilirubin is at least 2 times higher than the upper normal limit of the total bilirubin and the PT/INR ratio is higher 1.5.

In another aspect, the present disclosure relates to a method for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of a first liver biomarker and a second liver biomarker in a baseline sample obtained from the subject; and
c) comparing the subject baseline level of the first liver biomarker to the upper normal limit of the first liver biomarker, to the reference level of the first liver biomarker and to the subject reference level of the first liver biomarker;
d) comparing the subject baseline level of the second liver biomarker to the upper normal limit of the second liver biomarker, to the reference level of the second liver biomarker and to the subject reference level of the second liver biomarker;
e) perform close monitoring for a specific period of time if:
   a. the subject reference level of the first liver biomarker is higher than 1.5 times the upper normal limit of the first liver biomarker, or
   b. the subject reference level of the second liver biomarker is higher than 1.5 times the upper normal limit of the second liver biomarker,
   and
   c. the subject baseline level of the first liver biomarker is equal or higher than 3 times the subject reference level of the first liver biomarker, or equal or higher than 300 U/L, or
   d. the subject baseline level of the second liver biomarker is equal or higher than 3 times the subject reference level of the second liver biomarker, or equal or higher than 300 U/L,
      wherein the subject is at risk of experiencing an adverse side effect when:
(i) the panel of autoimmune disease biomarkers is present in a baseline sample obtained from the subject, or
(ii) the subject baseline level of the first liver biomarker increases or remains to at least 3 times the subject reference level of first liver biomarker and higher than 300 U/L, or
(iii) the subject baseline level of the second liver biomarker increases or remains to at least 3 times the subject reference level of second liver biomarker and higher than 300 U/L, or
(iv) the subject baseline level of the first liver biomarker increases or remains to at least 5 times the subject reference level of first liver biomarker, or
(v) the subject baseline level of the second liver biomarker increases or remains to at least 5 times the subject reference level of second liver biomarker, or
(vi) the subject baseline level of the first liver biomarker or the subject baseline level of the second liver biomarker increases or remains equal or higher than 500 U/L, or
(vii) the subject baseline level of the first liver biomarker increases or remains to at least 2 times the subject reference level of the first liver biomarker and the subject baseline level of at least one other liver biomarker has increased in the baseline sample, or
(viii) the subject baseline level of the second liver biomarker increases or remains to at least 2 times the subject reference level of second liver biomarker and the subject baseline level of at least one other liver biomarker has increased in the baseline sample, or
(ix) the subject baseline level of the first liver biomarker or the subject baseline level of the second liver biomarker increases or remains equal or higher than 300 U/L and the subject baseline level of at least one other liver biomarker has increased in the baseline sample, or
(x) the subject baseline level of the first liver biomarker increases or remains to at least 2 times the subject reference level of first liver biomarker, with the appearance of fatigue, nausea, vomiting, right upper quadrant pain or tenderness, fever, rash, and/or eosinophilia, or
(xi) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
(xii) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia.

According to the above-mentioned embodiment, the first liver biomarker is aspartate transaminase, the second liver biomarker is alanine transaminase and the at least one other liver biomarker is total bilirubin or PT/INR ratio.

Advantageously, the level of total bilirubin has increased and is at least 2 times higher than the upper normal limit of the total bilirubin. Advantageously, the PT/INR ratio has increased and is at least 1.08 times higher than the upper normal limit of the PT/INR ratio. Advantageously, the level of total bilirubin is at least 2 times higher than the upper normal limit of the total bilirubin or the PT/INR ratio is higher 1.5.

In some embodiments, the method for monitoring a subject's risk of experiencing an adverse side effect caused by lanifibranor treatment, comprises:
a) determining the presence of a panel of autoimmune disease biomarkers in a baseline sample obtained from the subject,
b) determining the subject baseline level of aspartate transaminase and a second liver biomarker in a baseline sample obtained from the subject; and
c) comparing the subject baseline level of aspartate transaminase to the upper normal limit of aspartate transaminase, to the reference level of aspartate transaminase and to the subject reference level of aspartate transaminase;
d) comparing the subject baseline level of alanine transaminase to the upper normal limit of alanine transaminase, to the reference level of alanine transaminase and to the subject reference level of alanine transaminase;
e) perform close monitoring for a specific period of time if:
   a. the subject reference level of aspartate transaminase is higher than 1.5 times the upper normal limit of aspartate transaminase, or
   b. the subject reference level of alanine transaminase is higher than 1.5 times the upper normal limit of alanine transaminase,
   and
   c. the subject baseline level of aspartate transaminase is equal or higher than 3 times the subject reference level of aspartate transaminase, or equal or higher than 300 U/L, or
   d. the subject baseline level of alanine transaminase is equal or higher than 3 times the subject reference level of alanine transaminase, or equal or higher than 300 U/L,
      wherein the subject is at risk of experiencing an adverse side effect when:
(i) the panel of autoimmune disease biomarkers is present in a baseline sample obtained from the subject, or
(ii) the subject baseline level of aspartate transaminase increases or remains to at least 3 times the subject reference level of aspartate transaminase and higher than 300 U/L, or
(iii) the subject baseline level of alanine transaminase increases or remains to at least 3 times the subject reference level of alanine transaminase and higher than 300 U/L, or
(iv) the subject baseline level of aspartate transaminase increases or remains to at least 5 times the subject reference level of aspartate transaminase, or
(v) the subject baseline level of alanine transaminase increases or remains to at least 5 times the subject reference level of alanine transaminase, or
(vi) the subject baseline level of aspartate transaminase or the subject baseline level of alanine transaminase increases or remains equal or higher than 500 U/L, or
(vii) the subject baseline level of aspartate transaminase increases or remains to at least 2 times the subject reference level of aspartate transaminase and the subject baseline level of total bilirubin or PT/INR ratio has increased in the baseline sample, or
(viii) the subject baseline level of alanine transaminase increases or remains to at least 2 times the subject reference level of alanine transaminase and the subject baseline level of total bilirubin or PT/INR ratio has increased in the baseline sample, or
(i) the subject baseline level of aspartate transaminase or the subject baseline level of alanine transaminase increases or remains equal or higher than 300 U/L and the subject baseline level of total bilirubin or PT/INR ratio has increased in the baseline sample, or
(ix) the subject baseline level of aspartate transaminase increases or remains to at least 2 times the subject reference level of aspartate transaminase, with the appearance of fatigue, nausea, vomiting, right upper quadrant pain or tenderness, fever, rash, and/or eosinophilia, or
(x) the subject baseline level of alanine transaminase increases or remains to at least 2 times the subject reference level of alanine transaminase, with the appearance of fatigue, nausea, vomiting, right upper quadrant pain or tenderness, fever, rash, and/or eosinophilia, or
(xi) the subject baseline level of aspartate transaminase or the subject baseline level of alanine transaminase increases or remains equal or higher than 300 U/L, with the appearance of fatigue, nausea, vomiting, right upper quadrant pain or tenderness, fever, rash, and/or eosinophilia.

In some embodiments, the specific period of time is of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, at least 96 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks. In some embodiments, the specific period of time is between at least 1 hour and 1 week, for example between at least 1 hour and at least 96 hours, for example between at least 24 hours and at least 96 hours, for example between at least 48 hours and at least 96 hours, for example between at least 48 hours and at least 72 hours.

In another aspect, the present disclosure relates to a method for monitoring a subject's risk of experiencing an adverse side effect caused by lanifibranor treatment, comprising:
a) determining the subject baseline level of at least one liver biomarker in a baseline sample obtained from the subject; and
b) comparing the subject baseline level of the at least one liver biomarker to the corresponding upper normal limit of the at least one liver biomarker, to the reference level of the at least one liver biomarker and to the subject reference level of the at least one liver biomarker;
   wherein the subject is at risk of experiencing an adverse side effect when:
(i) the subject baseline level of the at least one liver biomarker is higher than the reference level of said at least one liver biomarker; or
(ii) the subject baseline level of the at least one liver biomarker is higher than 300 U/L; or
(iii) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
(iv) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L.

In some embodiments, if the subject is not at risk of experiencing an adverse side effect, the method further comprises administering lanifibranor to the subject.

In another aspect, the present disclosure relates to a method for monitoring a subject's risk of experiencing an adverse side effect caused by lanifibranor treatment, comprising:
a) determining the subject baseline level of at least one liver biomarker in a baseline sample obtained from the subject; and
b) comparing the subject baseline level of the first liver biomarker to the upper normal limit of the first liver biomarker, to reference level of the first liver biomarker and to the subject reference level of the first liver biomarker; and
c) comparing the subject baseline level of the second liver biomarker to the upper normal limit of the second liver biomarker, to the reference level of the second liver biomarker and to the subject reference level of the second liver biomarker;
   wherein the subject is at risk of experiencing an adverse side effect when:
(i) the subject baseline level of the first liver biomarker is 10 times higher than the upper normal limit of the first liver biomarker, or is 2 times higher than reference level of the first liver biomarker, or is higher than 300 IU/L; or
(ii) the subject baseline level of the second liver biomarker is 10 times higher than the upper normal limit of the second liver biomarker, or is 2 times higher than reference level of the second liver biomarker, or is higher than 300 IU/L; or
(iii) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
(iv) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased.

In another aspect, the present disclosure relates to a method for monitoring a subject's risk of experiencing an adverse side effect caused by lanifibranor treatment, comprising:
a) determining the subject baseline level of a first liver biomarker and the subject baseline level of a second liver biomarker in a baseline sample obtained from the subject; and
b) comparing the subject baseline level of the first liver biomarker to the upper normal limit of the first liver biomarker and to the subject reference level of the first liver biomarker; and
c) comparing the subject baseline level of the second liver biomarker to the upper normal limit of the second liver biomarker and to the subject reference level of the second liver biomarker;
d) perform close monitoring for a specific period of time if:
   a. the subject reference level of the first liver biomarker is below than 1.5 times the upper normal limit of the first liver biomarker, or
   b. the subject reference level of the second liver biomarker is below than 1.5 times the upper normal limit of the second liver biomarker,
   and
   a. the subject baseline level of the first liver biomarker is equal or higher than 3 times the upper normal limit of the first liver biomarker but below than 5 times the upper normal limit of the first liver biomarker, or
   b. the subject baseline level of the second liver biomarker is equal or higher than 3 times the upper normal limit of the second liver biomarker but below than 5 times the upper normal limit of the second liver biomarker; or
   c. the subject baseline level of the first liver biomarker is equal or higher than 5 times the upper normal limit of the first liver biomarker, or
   d. the subject baseline level of the second liver biomarker is equal or higher than 5 times the upper normal limit of the second liver biomarker;
      wherein the subject is at risk of experiencing an adverse side effect when:
(i) the subject baseline level of the first liver biomarker increases or remains to at least the reference level of the first liver biomarker, or
(ii) the subject baseline level of the second liver biomarker increases or remains to at least the reference level of the second liver biomarker, or
(iii) the subject baseline level of the first liver biomarker is at least 5 times higher than the upper normal limit of the first liver biomarker, or
(iv) the subject baseline level of the second liver biomarker is at least 5 times higher than the upper normal limit of the second liver biomarker, or
(v) the subject baseline level of the first liver biomarker increases or remains to at least 3 times the upper normal limit of the first liver biomarker and the level of two others liver biomarkers has increased, or
(vi) the subject baseline level of the second liver biomarker increases or remains to at least 3 times the upper normal limit of the second liver biomarker and the level of two others liver biomarkers has increased, or
(vii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
(viii) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia.

In another aspect, the present disclosure relates to a method for monitoring a subject's risk of experiencing an adverse side effect caused by lanifibranor treatment, comprising:
a) determining the subject baseline level of a first liver biomarker and a second liver biomarker in a baseline sample obtained from the subject; and
b) comparing the subject baseline level of the first liver biomarker to the upper normal limit of the first liver biomarker, to the reference level of the first liver biomarker and to the subject reference level of the first liver biomarker;
c) comparing the subject baseline level of the second liver biomarker to the upper normal limit of the second liver biomarker, to the reference level of the second liver biomarker and to the subject reference level of the second liver biomarker;
d) performing close monitoring for a specific period of time if:
   a. the subject reference level of the first liver biomarker is higher than 1.5 times the upper normal limit of the first liver biomarker, or
   b. the subject reference level of the second liver biomarker is higher than 1.5 times the upper normal limit of the second liver biomarker,
   and
   a. the subject baseline level of the first liver biomarker is equal or higher than 3 times the subject reference level of the first liver biomarker, or equal or higher than 300 U/L, or
   b. the subject baseline level of the second liver biomarker is equal or higher than 3 times the subject reference level of the second liver biomarker, or equal or higher than 300 U/L,
      wherein the subject is at risk of experiencing an adverse side effect when:
(i) the subject baseline level of the first liver biomarker increases or remains to at least 3 times the subject reference level of first liver biomarker and higher than 300 U/L, or
(ii) the subject baseline level of the second liver biomarker increases or remains to at least 3 times the subject reference level of second liver biomarker and higher than 300 U/L, or
(iii) the subject baseline level of the first liver biomarker increases or remains to at least 5 times the subject reference level of first liver biomarker, or
(iv) the subject baseline level of the second liver biomarker increases or remains to at least 5 times the subject reference level of second liver biomarker, or
(v) the subject baseline level of the first liver biomarker or the subject baseline level of the second liver biomarker increases or remains equal or higher than 500 U/L, or
(vi) the subject baseline level of the first liver biomarker increases or remains to at least 2 times the subject reference level of the first liver biomarker and the level of at least one other liver biomarker has increased in the baseline sample, or
(vii) the subject baseline level of the second liver biomarker increases or remains to at least 2 times the subject reference level of second liver biomarker and the level of at least one other liver biomarker has increased in the baseline sample, or
(viii) the subject baseline level of the first liver biomarker or the subject baseline level of the second liver biomarker increases or remains equal or higher than 300 U/L, or
(ix) the subject baseline level of the first liver biomarker increases or remains to at least 2 times the subject reference level of first liver biomarker, with the appearance of fatigue, nausea, vomiting, right upper quadrant pain or tenderness, fever, rash, and/or eosinophilia, or
(x) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
(xi) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia.

In some embodiments, the specific period of time is of at least an 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, at least 96 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks. In some embodiments, the specific period of time is between at least 1 hour and 1 week, for example between at least 1 hour. In some embodiments, the specific period of time is between at least an hour and at least 96 hours, for example between at least 24 hours and at least 96 hours, for example between at least 48 hours and at least 96 hours, for example between at least 48 hours and at least 72 hours.

In some embodiments, the determining of the subject baseline level of at least one liver biomarker in a baseline sample obtained from the subject, are made at regular interval of time for a determined duration. In some embodiments, the regular interval of time is at least every 1 hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, at least every 1 month, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 1 year, at least every 2 years, at least every 3 years, at least every 4 years, at least every 5 years, at least every 6 years, at least every 7 years, at least every 8 years, at least every 9 years, at least every 10 years, at least every 11 years, at least every 12 years.

In some embodiments, the determined duration is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more. The determined duration, and a fortiori the end of the treatment, can vary depending upon the physical condition of the subject to be treated, a professional assessment of the medical situation, and other relevant factors.

In some embodiments, the determining of the subject baseline level of at least one liver biomarker in a baseline sample obtained from the subject is made at regular interval of time of at least every 1 hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, at least every 1 month, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 1 year, at least every 2 years, at least every 3 years, at least every 4 years, at least every 5 years, at least every 6 years, at least every 7 years, at least every 8 years, at least every 9 years, at least every 10 years, at least every 11 years, at least every 12 years for a determined duration of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more.

In some embodiments, the determining of the subject baseline level of at least one liver biomarker in a baseline sample obtained from the subject is made:
at regular interval of time of at least every 24 hours, at least every 36 hours, at least every 48 hours, at least every 60 hours, at least every 72 hours, at least every 1 day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, at least every 14 days, at least every 15 days, at least every 16 days, at least every 17 days, at least every 18 days, at least every 19 days, at least every 20 days, at least every 21 days, at least every 22 days, at least every 23 days, at least every 24 days, at least every 25 days, at least every 26 days, at least every 27 days, at least every 28 days, at least every 29 days, at least every 30 days, at least every 31 days, at least every 1 week, at least every 2 weeks, at least every 3 weeks, at least every 4 weeks, at least every 5 weeks, at least every 6 weeks, at least every 7 weeks, at least every 8 weeks, at least every 9 weeks, at least every 10 weeks, for a determined duration of at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years, and then
continued at regular interval of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least every 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least every 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least every 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least every 51 weeks, at least 52 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years, at least 3 years, at least 4 years, at least 5 years, at least 6 years, at least 7 years, at least 8 years, at least 9 years, at least 10 years, at least 11 years, at least 12 years or more.

In some embodiments, the determining of the subject baseline level of at least one liver biomarker in a baseline sample obtained from the subject is made at regular interval of time of 6 weeks for a determined duration of 10 months, and is then continued at regular interval of 3 months until the end of lanifibranor treatment.

In some embodiments, the determining of the subject baseline level of at least one liver biomarker in a baseline sample obtained from the subject is made at regular interval of time of 4 weeks for a determined duration of 10 months, and is then continued at regular interval of 3 months until the end of lanifibranor treatment.

In some embodiments, the determining of the subject baseline level of at least one liver biomarker in a baseline sample obtained from the subject is made at regular interval of time of 4 weeks for a determined duration of 10 months, and is then continued at regular interval of 6 weeks until the end of lanifibranor treatment.

In some embodiments, the determining of the subject baseline level of at least one liver biomarker in a baseline sample obtained from the subject is made at regular interval of time of 4 weeks for a determined duration of 48 weeks, and is then continued at regular interval of 6 weeks until the end of lanifibranor treatment.

In another aspect, the present disclosure provides a detection kit comprising means for detecting the panel of autoimmune disease biomarkers as set forth above and means for detecting the at least one liver biomarkers. In some embodiments, the kit comprises antibodies or peptides that are able to react with the panel of autoimmune disease biomarkers and the at least one liver biomarkers.

Aspects of the present disclosure are further illustrated by reference to the following, non-limiting embodiments.
1. A method of identifying a subject as eligible for treatment with lanifibranor, comprising:
   a) determining the presence or the absence of a panel of autoimmune disease biomarkers in a reference sample obtained from the subject,
   b) determining the subject reference level of at least one liver biomarker in a reference sample obtained from the subject,
   c) comparing the subject reference level of the at least one liver biomarker to the reference level of the at least one liver biomarker, and
   d) selecting the subject for treatment with lanifibranor if:
      (i) the panel of autoimmune disease biomarkers is absent in the reference sample, and
      (ii) the subject reference level of at least one liver biomarker is equal or below than the reference level of the at least one liver biomarker.
2. The method according to item 1, wherein the method further comprises administering an effective amount of lanifibranor to the subject selected for treatment with lanifibranor.
3. A method of identifying a subject as eligible for treatment with lanifibranor, comprising:
   a) obtaining a reference sample from the subject,
   b) determining the presence or the absence of a panel of autoimmune disease biomarkers in a reference sample obtained from the subject,
   c) determining the subject reference level of at least one liver biomarker in a reference sample obtained from the subject,
   d) comparing the subject reference level of the at least one liver biomarker to the reference level of the at least one liver biomarker, and
   e) selecting the subject for treatment with lanifibranor if:
      (i) the panel of autoimmune disease biomarkers is absent in the reference sample, and
      (ii) the subject reference level of at least one liver biomarker is equal or below than the reference level of the at least one liver biomarker;
   f) administering an effective amount of lanifibranor to the subject selected for treatment with lanifibranor.
4. A method of treating a subject with lanifibranor, comprising:
   a) determining the presence or the absence of a panel of autoimmune disease biomarkers in a reference sample obtained from the subject,
   b) determining the subject reference level of at least one liver biomarker in a reference sample obtained from the subject,
   c) comparing the subject reference level of the at least one liver biomarker to the reference level of the at least one liver biomarker, and
   d) administering lanifibranor to the patient if the patient has:
      (iii) the panel of autoimmune disease biomarkers is absent in the reference sample, and
      (iv) the subject reference level of at least one liver biomarker is equal or below than the reference level of the at least one liver biomarker.
5. A method of screening a subject at risk of adverse side effect from treatment with lanifibranor, comprising:
   a) determining the presence or the absence of a panel of autoimmune disease biomarkers in a reference sample obtained from the subject,
   b) determining the subject reference level of at least one liver biomarker in a reference sample obtained from the subject,
   c) comparing the subject reference level of the at least one liver biomarker to the reference level of the at least one liver biomarker,
      wherein the subject is at risk of adverse side effect from treatment with lanifibranor if:
(i) the panel of autoimmune disease biomarkers is present in the sample, and/or
(i) the subject reference level of at least one liver biomarker is higher than the reference level of the at least one liver biomarker.
6. The method according to item 5, wherein the method further comprises not administering lanifibranor to said subject.
7. A method for reducing the risk or occurrence of adverse effect in a subject suffering from liver disease, the method comprising:
   a) determining the presence or the absence of a panel of autoimmune disease biomarkers in a reference sample obtained from the subject,
   b) determining the subject reference level of at least one liver biomarker in a reference sample obtained from the subject,
   c) comparing the subject reference level of the at least one liver biomarker to the reference level of the at least one liver biomarker,
   d) administering to said subject an effective amount of lanifibranor if:
      (i) the panel of autoimmune disease biomarkers is absent in the reference sample, and
      (ii) the subject reference level of at least one liver biomarker is below than the reference level of the at least one liver biomarker.
8. A method for reducing the risk or occurrence of adverse effect caused by lanifibranor treatment in a subject suffering from liver disease, the method comprising:
   a) determining the presence or the absence of a panel of autoimmune disease biomarkers in a reference sample obtained from the subject,
   b) determining the subject reference level of at least one liver biomarker in a reference sample obtained from the subject,
   c) comparing the subject reference level of the at least one liver biomarker to the reference level of the at least one liver biomarker,
   d) wherein the risk or occurrence of adverse effect caused by lanifibranor treatment in a subject suffering from liver disease is reduced when:
      (i) the panel of autoimmune disease biomarkers is absent in the reference sample, and
      (ii) the subject reference level of at least one liver biomarker is below than the reference level of the at least one liver biomarker.
9. A method for monitoring a subject's risk of experiencing an adverse side effect caused by lanifibranor treatment, comprising:
   a) determining the presence of a panel of autoimmune disease biomarkers in a baseline sample obtained from the subject,
   b) determining the subject baseline level of at least one liver biomarker in a baseline sample obtained from the subject; and
   c) comparing the subject baseline level of the at least one liver biomarker to the corresponding upper normal limit of the at least one liver biomarker, to the reference level of the at least one liver biomarker and to the subject reference level of the at least one liver biomarker;
      wherein the subject is at risk of experiencing the adverse side effect when:
(i) the panel of autoimmune disease biomarkers is present in a baseline sample obtained from the subject, and/or
(ii) the subject baseline level of the at least one liver biomarker is higher than the reference level of said at least one liver biomarker; or
(iii) the subject baseline level of the at least one liver biomarker is higher than 300 U/L; or
(iv) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
(v) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L.
10. The method of item 9, wherein if the subject is not at risk of experiencing an adverse side effect, the method further comprises administering lanifibranor to the subject.
11. A method for monitoring a subject's risk of experiencing an adverse side effect caused by lanifibranor treatment, comprising:
   a) determining the presence of a panel of autoimmune disease biomarkers in a baseline sample obtained from the subject,
   b) determining the subject baseline level of at least one liver biomarker in a baseline sample obtained from the subject; and
   c) comparing the subject baseline level of the first liver biomarker to the upper normal limit of the first liver biomarker, to reference level of the first liver biomarker and to the subject reference level of the first liver biomarker; and
   d) comparing the subject baseline level of the second liver biomarker to the upper normal limit of the second liver biomarker, to the reference level of the second liver biomarker and to the subject reference level of the second liver biomarker;
wherein subject is at risk of experiencing an adverse side effect when:
   (i) the panel of autoimmune disease biomarkers is present in a baseline sample obtained from the subject, and/or
   (ii) the subject baseline level of the first liver biomarker is 10 times higher than the upper normal limit of the first liver biomarker, or is 2 times higher than reference level of the first liver biomarker, or is higher than 300 IU/L; or
   (iii) the subject baseline level of the second liver biomarker is 10 times higher than the upper normal limit of the second liver biomarker, or is 2 times higher than reference level of the second liver biomarker, or is higher than 300 IU/L; or
   (iv) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
   (v) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased.
12. A method for monitoring a subject's risk of experiencing an adverse side effect caused by lanifibranor treatment, comprising:
   a) determining the presence of a panel of autoimmune disease biomarkers in a baseline sample obtained from the subject,
   b) determining the subject baseline level of a first liver biomarker and the subject baseline level of a second liver biomarker in a baseline sample obtained from the subject; and
   c) comparing the subject baseline level of the first liver biomarker to the upper normal limit of the first liver biomarker and to the subject reference level of the first liver biomarker; and
   d) comparing the subject baseline level of the second liver biomarker to the upper normal limit of the second liver biomarker and to the subject reference level of the second liver biomarker;
   e) performing close monitoring for a specific period of time if:
      a. the subject reference level of the first liver biomarker is below than 1.5 times the upper normal limit of the first liver biomarker, or
      b. the subject reference level of the second liver biomarker is below than 1.5 times the upper normal limit of the second liver biomarker,
      and
      a. the subject baseline level of the first liver biomarker is equal or higher than 3 times the upper normal limit of the first liver biomarker but below than 5 times the upper normal limit of the first liver biomarker, or
      b. the subject baseline level of the second liver biomarker is equal or higher than 3 times the upper normal limit of the second liver biomarker but below than 5 times the upper normal limit of the second liver biomarker; or
      c. the subject baseline level of the first liver biomarker is equal or higher than 5 times the upper normal limit of the first liver biomarker, or
      d. the subject baseline level of the second liver biomarker is equal or higher than 5 times the upper normal limit of the second liver biomarker;
wherein the subject is at risk of experiencing an adverse side effect when:
   (i) the panel of autoimmune disease biomarkers is present in a baseline sample obtained from the subject, or
   (ii) the subject baseline level of the first liver biomarker increases or remains to at least the reference level of the first liver biomarker, or
   (iii) the subject baseline level of the second liver biomarker increases or remains to at least the reference level of the second liver biomarker, or
   (iv) the subject baseline level of the first liver biomarker is at least 5 times higher than the upper normal limit of the first liver biomarker, or
   (v) the subject baseline level of the second liver biomarker is at least 5 times higher than the upper normal limit of the second liver biomarker, or
   (vi) the subject baseline level of the first liver biomarker increases or remains to at least 3 times the upper normal limit of the first liver biomarker and the level of two others liver biomarkers has increased, or
   (vii) the subject baseline level of the second liver biomarker increases or remains to at least 3 times the upper normal limit of the second liver biomarker and the level of two others liver biomarkers has increased, or
   (viii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
   (ix) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia.
13. A method for monitoring a subject's risk of experiencing an adverse side effect caused by lanifibranor treatment, comprising:
   a) determining the presence of a panel of autoimmune disease biomarkers in a baseline sample obtained from the subject,
   b) determining the subject baseline level of a first liver biomarker and a second liver biomarker in a baseline sample obtained from the subject; and
   c) comparing the subject baseline level of the first liver biomarker to the upper normal limit of the first liver biomarker, to the reference level of the first liver biomarker and to the subject reference level of the first liver biomarker;
   d) comparing the subject baseline level of the second liver biomarker to the upper normal limit of the second liver biomarker, to the reference level of the second liver biomarker and to the subject reference level of the second liver biomarker;
   e) performing close monitoring for a specific period of time if:
      a. the subject reference level of the first liver biomarker is higher than 1.5 times the upper normal limit of the first liver biomarker, or
      b. the subject reference level of the second liver biomarker is higher than 1.5 times the upper normal limit of the second liver biomarker,
      and
      a. the subject baseline level of the first liver biomarker is equal or higher than 3 times the subject reference level of the first liver biomarker, or equal or higher than 300 U/L, or
      b. the subject baseline level of the second liver biomarker is equal or higher than 3 times the subject reference level of the second liver biomarker, or equal or higher than 300 U/L,
wherein the subject is at risk of experiencing the adverse side effect when:
   (i) the panel of autoimmune disease biomarkers is present in a baseline sample obtained from the subject, or
   (ii) the subject baseline level of the first liver biomarker increases or remains to at least 3 times the subject reference level of first liver biomarker and higher than 300 U/L, or
   (iii) the subject baseline level of the second liver biomarker increases or remains to at least 3 times the subject reference level of second liver biomarker and higher than 300 U/L, or
   (iv) the subject baseline level of the first liver biomarker increases or remains to at least 5 times the subject reference level of first liver biomarker, or
   (v) the subject baseline level of the second liver biomarker increases or remains to at least 5 times the subject reference level of second liver biomarker, or
   (vi) the subject baseline level of the first liver biomarker or the subject baseline level of the second liver biomarker increases or remains equal or higher than 500 U/L, or
   (vii) the subject baseline level of the first liver biomarker increases or remains to at least 2 times the subject reference level of the first liver biomarker and the level of at least one other liver biomarker has increased in the baseline sample, or
   (viii) the subject baseline level of the second liver biomarker increases or remains to at least 2 times the subject reference level of second liver biomarker and the level of at least one other liver biomarker has increased in the baseline sample, or
   (ix) the subject baseline level of the first liver biomarker or the subject baseline level of the second liver biomarker increases or remains equal or higher than 300 U/L and the subject baseline level of at least one other liver biomarker has increased in the baseline sample, or
   (x) the subject baseline level of the first liver biomarker increases or remains to at least 2 times the subject reference level of first liver biomarker, with the appearance of fatigue, nausea, vomiting, right upper quadrant pain or tenderness, fever, rash, and/or eosinophilia, or
   (xi) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
   (xii) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia.
14. A method for monitoring a subject's risk of experiencing an adverse side effect caused by lanifibranor treatment, comprising:
   a) determining the presence of a panel of autoimmune disease biomarkers in a baseline sample obtained from the subject,
   b) determining the subject baseline level of a first liver biomarker and a second liver biomarker in a baseline sample obtained from the subject; and
   c) comparing the subject baseline level of the first liver biomarker to the upper normal limit of the first liver biomarker or to the subject reference level of the first liver biomarker;
   d) comparing the subject baseline level of the second liver biomarker to the upper normal limit of the second liver biomarker or to the subject reference level of the second liver biomarker;
   e) performing close monitoring for a specific period of time if:
      a. the subject reference level of the first liver biomarker is higher than 1.5 times the upper normal limit of the first liver biomarker, or
      b. the subject reference level of the second liver biomarker is higher than 1.5 times the upper normal limit of the second liver biomarker,
      and
      a. the subject baseline level of the first liver biomarker is equal or higher than 3 times the subject reference level of the first liver biomarker, or equal or higher than 300 U/L, or
      b. the subject baseline level of the second liver biomarker is equal or higher than 3 times the subject reference level of the second liver biomarker, or equal or higher than 300 U/L,
wherein the subject is at risk of experiencing an adverse side effect when:
   (i) the panel of autoimmune disease biomarkers is present in a baseline sample obtained from the subject, or
   (ii) the subject baseline level of aspartate transaminase increases or remains to at least 3 times the subject reference level of aspartate transaminase and higher than 300 U/L, or
   (iii) the subject baseline level of alanine transaminase increases or remains to at least 3 times the subject reference level of alanine transaminase and higher than 300 U/L, or
   (iv) the subject baseline level of aspartate transaminase increases or remains to at least 5 times the subject reference level of aspartate transaminase or,
   (v) the subject baseline level of alanine transaminase increases or remains to at least 5 times the subject reference level of alanine transaminase or,
   (vi) the subject baseline level of aspartate transaminase or the subject baseline level of alanine transaminase increases or remains equal or higher than 500 U/L or,
   (vii) the subject baseline level of aspartate transaminase increases or remains to at least 2 times the subject reference level of aspartate transaminase and the subject baseline level of total bilirubin or PT/INR ratio has increased in the baseline sample, or
   (viii) the subject baseline level of alanine transaminase increases or remains to at least 2 times the subject reference level of alanine ransaminase and the subject baseline level of total bilirubin or PT/INR ratio has increased in the baseline sample, or
   (ix) the subject baseline level of aspartate transaminase or the subject baseline level of alanine transaminase increases or remains equal or higher than 300 U/L L and the subject baseline level of total bilirubin or PT/INR ratio has increased in the baseline sample, or
   (x) the subject baseline level of aspartate transaminase increases or remains to at least 2 times the subject reference level of aspartate transaminase, with the appearance of fatigue, nausea, vomiting, right upper quadrant pain or tenderness, fever, rash, and/or eosinophilia, or
   (xi) the subject baseline level of alanine transaminase increases or remains to at least 2 times the subject reference level of alanine transaminase, with the appearance of fatigue, nausea, vomiting, right upper quadrant pain or tenderness, fever, rash, and/or eosinophilia, or
   (xii) the subject baseline level of aspartate transaminase or the subject baseline level of alanine transaminase increases or remains equal or higher than 300 U/L, with the appearance of fatigue, nausea, vomiting, right upper quadrant pain or tenderness, fever, rash, and/or eosinophilia.
15. The method according to any one of items 1 to 14, wherein the panel of autoimmune disease biomarkers is indicative for predisposition to an autoimmune disease.
16. The method according to any one of items 1 to 15, wherein the panel of autoimmune disease biomarkers comprises at least one autoimmune disease biomarkers selected from the group comprising: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, anti-liver cytosol type 1 antibody and a combination thereof.
17. The method according to items 1 to 16, wherein the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.
18. The method according to items 16 to 17, wherein the anti-nuclear antibody comprises a panel of different antibodies selected from the group comprising: anti-double-stranded DNA (dsDNA) antibody, anti-histone antibody, anti-chromatin antibody, anti-Scl-70 antibody, anti-smith antibody, anti-Sm/RNP antibody, anti-Jo-1 antibody, anti-centromeric protein antibody, anti-SSA-52 antibody, anti-SSA-60 antibody, anti-SSB antibody, anti-RNA polymerase III antibody, anti-Ku antibody, anti-CENP-A antibody, anti-CENP-B antibody, anti-fibrillarin antibody, anti-PM/Scl-100 antibody, anti-Th/To antibody, anti-Sp-100 antibody, anti-PML antibody, anti-NXP-2 antibody, anti-MORC3 antibody, anti-MJ antibody and a combination thereof.
19. The method according to any one of items 1 to 18, wherein the at least one liver biomarker is selected from the group comprising: aspartate transaminase, alanine transaminase, alkaline phosphatase, gamma-glutamyl transferase, bilirubin, prothrombin time test with an international normalized ratio and a combination thereof.
20. The method according to any one of items 1 to 19, wherein the reference sample is a sample obtained from the subject prior to the first administration of lanifibranor treatment and wherein the baseline sample is a sample obtained from the same subject after administration of lanifibranor treatment.
21. The method according to any one of items 1 to 20, wherein the reference sample and the baseline sample are from the same source and are selected from saliva sample, hair sample, skin sample, tissue sample, sputum sample, blood sample, plasma sample, serum sample, vitreal sample, cerebrospinal fluid sample, urine sample, sperm sample and cells sample.
22. The method according to any one of items 1 to 21, wherein the subject is suffering from a liver disease selected among: liver fibrosis, liver steatosis, fatty liver disease, acute decompensation, acute decompensation of cirrhosis, acute on chronic liver failure (ACLF), acute liver failure (ALF), decompensated cirrhosis, compensated cirrhosis, non-alcoholic fatty liver disease (NAFLD), non-alcoholic steatohepatitis (NASH), metabolic-associated fatty liver disease (MAFLD), metabolic-associated steatohepatitis (MASH), diabetes, primary sclerosing cholangitis (PSC), hepatocarcinoma, non-alcoholic steatohepatitis with moderate to advanced liver fibrosis (consistent with stages F2 to F3 fibrosis), metabolic-associated steatohepatitis with moderate to advanced liver fibrosis (consistent with stages F2 to F3 fibrosis), non-cirrhotic non-alcoholic steatohepatitis with moderate to advanced liver fibrosis (consistent with stages F2 to F3 fibrosis), non-cirrhotic metabolic-associated steatohepatitis with moderate to advanced liver fibrosis (consistent with stages F2 to F3 fibrosis) and liver cirrhosis.
23. The method according to item 22, wherein the liver disease is associated with diabetes, and in particular with type 2 diabetes.
24. The method according to any one of items 1 to 23, wherein the autoimmune disease is selected from the group comprising: autoimmune hepatitis, dermatomyositis, juvenile idiopathic arthritis, mixed connective tissue disease, primary biliary cholangitis, polymyositis, systemic autoimmune rheumatic disease, Sjögren syndrome, systemic lupus erythematosus, systemic sclerosis, undifferentiated connective tissue disease, autoimmune liver disease and autoimmune thyroid disease.
25. The method according to item 23 wherein the autoimmune liver disease is selected from the group comprising: autoimmune hepatitis, autoimmune cholangitis, autoimmune cholangiopathy and overlap syndrome.
26. The method according to any one of items 1 to 25, wherein the effective amount of lanifibranor comprises from 0.5 to 1200 mg of lanifibranor once a day.
27. A method for monitoring a subject's risk of experiencing an adverse side effect caused by lanifibranor treatment, comprising:
   a) determining the subject baseline level of at least one liver biomarker in a baseline sample obtained from the subject; and
   b) comparing the subject baseline level of the at least one liver biomarker to the corresponding upper normal limit of the at least one liver biomarker, to the reference level of the at least one liver biomarker and to the subject reference level of the at least one liver biomarker;
   wherein the subject is at risk of experiencing an adverse side effect when:
      (i) the subject baseline level of the at least one liver biomarker is higher than the reference level of said at least one liver biomarker; or
      (ii) the subject baseline level of the at least one liver biomarker is higher than 300 U/L; or
      (iii) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
      (iv) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L.
28. The method of item 27, wherein if the subject is not at risk of experiencing the adverse side effect, the method further comprises administering lanifibranor to the subject.
29. A method for monitoring a subject's risk of experiencing an adverse side effect caused by lanifibranor treatment, comprising:
   a) determining the subject baseline level of at least one liver biomarker in a baseline sample obtained from the subject; and
   b) comparing the subject baseline level of the first liver biomarker to the upper normal limit of the first liver biomarker, to reference level of the first liver biomarker and to the subject reference level of the first liver biomarker; and
   c) comparing the subject baseline level of the second liver biomarker to the upper normal limit of the second liver biomarker, to the reference level of the second liver biomarker and to the subject reference level of the second liver biomarker;
wherein the subject is at risk of experiencing an adverse side effect when:
   (i) the subject baseline level of the first liver biomarker is 10 times higher than the upper normal limit of the first liver biomarker, or is 2 times higher than reference level of the first liver biomarker, or is higher than 300 IU/L; or
   (ii) the subject baseline level of the second liver biomarker is 10 times higher than the upper normal limit of the second liver biomarker, or is 2 times higher than reference level of the second liver biomarker, or is higher than 300 IU/L; or
   (iii) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
   (iv) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased.
30. A method for monitoring a subject's risk of experiencing an adverse side effect caused by lanifibranor treatment, comprising:
   a) determining the subject baseline level of a first liver biomarker and the subject baseline level of a second liver biomarker in a baseline sample obtained from the subject; and
   b) comparing the subject baseline level of the first liver biomarker to the upper normal limit of the first liver biomarker and to the subject reference level of the first liver biomarker; and
   c) comparing the subject baseline level of the second liver biomarker to the upper normal limit of the second liver biomarker and to the subject reference level of the second liver biomarker;
   d) performing close monitoring for a specific period of time if:
      a. the subject reference level of the first liver biomarker is below than 1.5 times the upper normal limit of the first liver biomarker, or
      b. the subject reference level of the second liver biomarker is below than 1.5 times the upper normal limit of the second liver biomarker,
      and
      c. the subject baseline level of the first liver biomarker is equal or higher than 3 times the upper normal limit of the first liver biomarker but below than 5 times the upper normal limit of the first liver biomarker, or
      d. the subject baseline level of the second liver biomarker is equal or higher than 3 times the upper normal limit of the second liver biomarker but below than 5 times the upper normal limit of the second liver biomarker; or
      e. the subject baseline level of the first liver biomarker is equal or higher than 5 times the upper normal limit of the first liver biomarker, or
      f. the subject baseline level of the second liver biomarker is equal or higher than 5 times the upper normal limit of the second liver biomarker;
wherein the subject is at risk of experiencing the adverse side effect when:
   (i) the subject baseline level of the first liver biomarker increases or remains to at least the reference level of the first liver biomarker, or
   (ii) the subject baseline level of the second liver biomarker increases or remains to at least the reference level of the second liver biomarker, or
   (iii) the subject baseline level of the first liver biomarker is at least 5 times higher than the upper normal limit of the first liver biomarker, or
   (iv) the subject baseline level of the second liver biomarker is at least 5 times higher than the upper normal limit of the second liver biomarker, or
   (v) the subject baseline level of the first liver biomarker increases or remains to at least 3 times the upper normal limit of the first liver biomarker and the level of two others liver biomarkers has increased, or
   (vi) the subject baseline level of the second liver biomarker increases or remains to at least 3 times the upper normal limit of the second liver biomarker and the level of two others liver biomarkers has increased, or
   (vii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
   (viii) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia.
31. A method for monitoring a subject's risk of experiencing an adverse side effect caused by lanifibranor treatment, comprising:
   a) determining the subject baseline level of a first liver biomarker and a second liver biomarker in a baseline sample obtained from the subject; and
   b) comparing the subject baseline level of the first liver biomarker to the upper normal limit of the first liver biomarker, to the reference level of the first liver biomarker and to the subject reference level of the first liver biomarker;
   c) comparing the subject baseline level of the second liver biomarker to the upper normal limit of the second liver biomarker, to the reference level of the second liver biomarker and to the subject reference level of the second liver biomarker;
   d) perform close monitoring for a specific period of time if:
      a. the subject reference level of the first liver biomarker is higher than 1.5 times the upper normal limit of the first liver biomarker, or
      b. the subject reference level of the second liver biomarker is higher than 1.5 times the upper normal limit of the second liver biomarker,
      and
      c. the subject baseline level of the first liver biomarker is equal or higher than 3 times the subject reference level of the first liver biomarker, or equal or higher than 300 U/L, or
      d. the subject baseline level of the second liver biomarker is equal or higher than 3 times the subject reference level of the second liver biomarker, or equal or higher than 300 U/L,
wherein the subject is at risk of experiencing the adverse side effect when:
   (i) the subject baseline level of the first liver biomarker increases or remains to at least 3 times the subject reference level of first liver biomarker and higher than 300 U/L, or
   (ii) the subject baseline level of the second liver biomarker increases or remains to at least 3 times the subject reference level of second liver biomarker and higher than 300 U/L, or
   (iii) the subject baseline level of the first liver biomarker increases or remains to at least 5 times the subject reference level of first liver biomarker, or
   (iv) the subject baseline level of the second liver biomarker increases or remains to at least 5 times the subject reference level of second liver biomarker, or
   (v) the subject baseline level of the first liver biomarker or the subject baseline level of the second liver biomarker increases or remains equal or higher than 500 U/L, or
   (vi) the subject baseline level of the first liver biomarker increases or remains to at least 2 times the subject reference level of the first liver biomarker and the level of at least one other liver biomarker has increased in the baseline sample, or
   (vii) the subject baseline level of the second liver biomarker increases or remains to at least 2 times the subject reference level of second liver biomarker and the level of at least one other liver biomarker has increased in the baseline sample, or
   (viii) the subject baseline level of the first liver biomarker or the subject baseline level of the second liver biomarker increases or remains equal or higher than 300 U/L, or
   (ix) the subject baseline level of the first liver biomarker increases or remains to at least 2 times the subject reference level of first liver biomarker, with the appearance of fatigue, nausea, vomiting, right upper quadrant pain or tenderness, fever, rash, and/or eosinophilia, or
   (x) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
   (xi) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia.
32. The method according to any one of items 1 to 31, wherein the determining of the presence of a panel of autoimmune disease biomarkers and/or the determining of the subject baseline level of at least one liver biomarker in a baseline sample obtained from the subject, are made at regular interval of time for a determined duration.

1. A method
1) of identifying a subject as eligible for treatment with lanifibranor, comprising:
a) determining the presence or the absence of a panel of autoimmune disease biomarkers in a reference sample obtained from the subject;
b) determining the subject reference level of at least one liver biomarker in a reference sample obtained from the subject, wherein the at least one liver biomarker is selected from the group consisting of: aspartate transaminase, alanine transaminase, alkaline phosphatase, gamma-glutamyl transferase, bilirubin, prothrombin time test with an international normalized ratio and a combination thereof;
c) comparing the subject reference level of the at least one liver biomarker to the reference level of the at least one liver biomarker; and
d) selecting the subject for treatment with lanifibranor if:
(i) the panel of autoimmune disease biomarkers is absent in the reference sample, and
(ii) the subject reference level of at least one liver biomarker is equal or below than the reference level of the at least one liver biomarker;
or
2) of screening a subject at risk of adverse side effect from treatment with lanifibranor, comprising:
a) determining the presence or the absence of a panel of autoimmune disease biomarkers in a reference sample obtained from the subject:
b) determining the subject reference level of at least one liver biomarker in a reference sample obtained from the subject, wherein the at least one liver biomarker is selected from the group consisting of: aspartate transaminase, alanine transaminase, alkaline phosphatase, gamma-glutamyl transferase, bilirubin, prothrombin time test with an international normalized ratio and a combination thereof:
c) comparing the subject reference level of the at least one liver biomarker to the reference level of the at least one liver biomarker;
wherein the subject is at risk of adverse side effect from treatment with lanifibranor if:
(i) the panel of autoimmune disease biomarkers is present in the sample, and/or
(ii) the subject reference level of at least one liver biomarker is higher than the reference level of the at least one liver biomarker;
or
3) for reducing the risk or occurrence of adverse effect in a subject suffering from liver disease, the method comprising:
a) determining the presence or the absence of a panel of autoimmune disease biomarkers in a reference sample obtained from the subject;
b) determining the subject reference level of at least one liver biomarker in a reference sample obtained from the subject, wherein the at least one liver biomarker is selected from the group consisting of: aspartate transaminase, alanine transaminase, alkaline phosphatase, gamma-glutamyl transferase, bilirubin, prothrombin time test with an international normalized ratio and a combination thereof;
c) comparing the subject reference level of the at least one liver biomarker to the reference level of the at least one liver biomarker;
d) administering to said subject an effective amount of lanifibranor if:
(i) the panel of autoimmune disease biomarkers is absent in the reference sample, and
(ii) the subject reference level of at least one liver biomarker is below than the reference level of the at least one liver biomarker;
or
4) for monitoring a subject's risk of experiencing an adverse side effect caused by lanifibranor treatment, comprising:
a) determining the presence of a panel of autoimmune disease biomarkers in a baseline sample obtained from the subject;
b) determining the subject baseline level of at least one liver biomarker in a baseline sample obtained from the subject, wherein the at least one liver biomarker is selected from the group consisting of: aspartate transaminase, alanine transaminase, alkaline phosphatase, gamma-glutamyl transferase, bilirubin, prothrombin time test with an international normalized ratio and a combination thereof; and
c) comparing the subject baseline level of the at least one liver biomarker to the corresponding upper normal limit of the at least one liver biomarker, to the reference level of the at least one liver biomarker and to the subject reference level of the at least one liver biomarker;
wherein the subject is at risk of experiencing an adverse side effect when;
(i) the panel of autoimmune disease biomarkers is present in a baseline sample obtained from the subject, and/or
(ii) the subject baseline level of the at least one liver biomarker is higher than the reference level of said at least one liver biomarker: or
(iii) the subject baseline level of the at least one liver biomarker is higher than 300 U/L; or
(iv) the subject reference level of said at least one liver biomarker is below than 1.5 times the upper normal limit of the said at least one liver biomarker and the subject baseline level of the at least one liver biomarker is equal or higher than 3 times the upper normal limit of said at least one liver biomarker; or
(v) the subject reference level of said at least one liver biomarker is equal or higher than 1.5 times the upper normal limit of the said at least one liver biomarker and the subject baseline level of the at least one liver biomarker is equal or higher than 3 times the subject reference level of said at least one liver biomarker or higher than 300 IU/L;
or
5) for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of at least one liver biomarker in a baseline sample obtained from the subject; and
c) comparing the subject baseline level of a first liver biomarker to the upper normal limit of the first liver biomarker, to reference level of the first liver biomarker and to the subject reference level of the first liver biomarker; and
d) comparing the subject baseline level of a second liver biomarker to the upper normal limit of the second liver biomarker, to the reference level of the second liver biomarker and to the subject reference level of the second liver biomarker;
wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof;
wherein the subject is at risk of experiencing an adverse side effect when:
(i) the panel of autoimmune disease biomarkers is present in a baseline sample obtained from the subject, and/or
(ii) the subject baseline level of the first liver biomarker is 10 times higher than the upper normal limit of the first liver biomarker, or is 2 times higher than reference level of the first liver biomarker, or is higher than 300 IU/L; or
(iii) the subject baseline level of the second liver biomarker is 10 times higher than the upper normal limit of the second liver biomarker, or is 2 times higher than reference level of the second liver biomarker, or is higher than 300 IU/L; or
(iv) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
(v) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased;
or
6) for monitoring a subject's risk of experiencing an adverse side effect caused by lanifibranor treatment, comprising:
a) determining the presence of a panel of autoimmune disease biomarkers in a baseline sample obtained from the subject;
b) determining the subject baseline level of a first liver biomarker and the subject baseline level of a second liver biomarker in a baseline sample obtained from the subject, wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof;
c) comparing the subject baseline level of the first liver biomarker to the upper normal limit of the first liver biomarker and to the subject reference level of the first liver biomarker;
d) comparing the subject baseline level of the second liver biomarker to the upper normal limit of the second liver biomarker and to the subject reference level of the second liver biomarker; and
c) performing close monitoring for a specific period of time if;
a. the subject reference level of the first liver biomarker is below than 1.5 times the upper normal limit of the first liver biomarker, or
b. the subject reference level of the second liver biomarker is below than 1.5 times the upper normal limit of the second liver biomarker
and
c. the subject baseline level of the first liver biomarker is equal or higher than 3 times the upper normal limit of the first liver biomarker but below than 5 times the upper normal limit of the first liver biomarker, or
d. the subject baseline level of the second liver biomarker is equal or higher than 3 times the upper normal limit of the second liver biomarker but below than 5 times the upper normal limit of the second liver biomarker: or
c. the subject baseline level of the first liver biomarker is equal or higher than 5 times the upper normal limit of the first liver biomarker, or
f. the subject baseline level of the second liver biomarker is equal or higher than 5 times the upper normal limit of the second liver biomarker;
wherein the subject is at risk of experiencing an adverse side effect when:
(i) the panel of autoimmune disease biomarkers is present in a baseline sample obtained from the subject, or
(ii) the subject baseline level of the first liver biomarker increases or remains to at least the reference level of the first liver biomarker, or
(iii) the subject baseline level of the second liver biomarker increases or remains to at least the reference level of the second liver biomarker, or
(iv) the subject baseline level of the first liver biomarker is at least 5 times higher than the upper normal limit of the first liver biomarker, or
(v) the subject baseline level of the second liver biomarker is at least 5 times higher than the upper normal limit of the second liver biomarker, or
(vi) the subject baseline level of the first liver biomarker increases or remains to at least 3 times the upper normal limit of the first liver biomarker and the level of two others liver biomarkers has increased, or
(vii) the subject baseline level of the second liver biomarker increases or remains to at least 3 times the upper normal limit of the second liver biomarker and the level of two others liver biomarkers has increased, or
(viii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
(ix) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia;
or
7) for monitoring a subject's risk of experiencing an adverse side effect caused by lanifibranor treatment, comprising:
a) determining the presence of a panel of autoimmune disease biomarkers in a baseline sample obtained from the subject,
b) determining the subject baseline level of a first liver biomarker and a second liver biomarker in a baseline sample obtained from the subject, wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof;
c) comparing the subject baseline level of the first liver biomarker to the upper normal limit of the first liver biomarker, to the reference level of the first liver biomarker and to the subject reference level of the first liver biomarker;
d) comparing the subject baseline level of the second liver biomarker to the upper normal limit of the second liver biomarker, to the reference level of the second liver biomarker and to the subject reference level of the second liver biomarker; and
e) performing close monitoring for a specific period of time if:
a. the subject reference level of the first liver biomarker is higher than 1.5 times the upper normal limit of the first liver biomarker, or
b. the subject reference level of the second liver biomarker is higher than 1.5 times the upper normal limit of the second liver biomarker,
and
c. the subject baseline level of the first liver biomarker is equal or higher than 3 times the subject reference level of the first liver biomarker, or equal or higher than 300 U/L, or
d. the subject baseline level of the second liver biomarker is equal or higher than 3 times the subject reference level of the second liver biomarker, or equal or higher than 300 U/L,
wherein the subject is at risk of experiencing an adverse side effect when:
(i) the panel of autoimmune disease biomarkers is present in a baseline sample obtained from the subject, or
(ii) the subject baseline level of the first liver biomarker increases or remains to at least 3 times the subject reference level of first liver biomarker and higher than 300 U/L, or
(iii) the subject baseline level of the second liver biomarker increases or remains to at least 3 times the subject reference level of second liver biomarker and higher than 300 U/L, or
(iv) the subject baseline level of the first liver biomarker increases or remains to at least 5 times the subject reference level of first liver biomarker, or
(v) the subject baseline level of the second liver biomarker increases or remains to at least 5 times the subject reference level of second liver biomarker, or
(vi) the subject baseline level of the first liver biomarker or the subject baseline level of the second liver biomarker increases or remains equal or higher than 500 U/L, or
(vii) the subject baseline level of the first liver biomarker increases or remains to at least 2 times the subject reference level of the first liver biomarker and the level of at least one other liver biomarker has increased in the baseline sample, or
(viii) the subject baseline level of the second liver biomarker increases or remains to at least 2 times the subject reference level of second liver biomarker and the level of at least one other liver biomarker has increased in the baseline sample, or
(ix) the subject baseline level of the first liver biomarker or the subject baseline level of the second liver biomarker increases or remains equal or higher than 300 U/L, or
(x) the subject baseline level of the first liver biomarker increases or remains to at least 2 times the subject reference level of first liver biomarker, with the appearance of fatigue. nausea, vomiting, right upper quadrant pain or tenderness, fever, rash, and/or eosinophilia, or
(xi) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
(xii) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia;
or
8) for monitoring a subject's risk of experiencing an adverse side effect caused by lanifibranor treatment, comprising:
a) determining the subject baseline level of at least one liver biomarker in a baseline sample obtained from the subject; and
b) comparing the subject baseline level of a first liver biomarker to the upper normal limit of the first liver biomarker, to reference level of the first liver biomarker and to the subject reference level of the first liver biomarker; and
c) comparing the subject baseline level of a second liver biomarker to the upper normal limit of the second liver biomarker, to the reference level of the second liver biomarker and to the subject reference level of the second liver biomarker;
wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof;
wherein the subject is at risk of experiencing an adverse side effect when:
(i) the panel of autoimmune disease biomarkers is present in a baseline sample obtained from the subject, and/or
(ii) the subject baseline level of the first liver biomarker is 10 times higher than the upper normal limit of the first liver biomarker, or is 2 times higher than reference level of the first liver biomarker, or is higher than 300 IU/L; or
(iii) the subject baseline level of the second liver biomarker is 10 times higher than the upper normal limit of the second liver biomarker, or is 2 times higher than reference level of the second liver biomarker, or is higher than 300 IU/L; or
(iv) the subject baseline level of the first liver biomarker is at least 3 times higher than the upper normal limit of said first liver biomarker or the subject baseline level of first liver biomarker is at least 3 times higher than the subject reference level of the first liver biomarker and the subject baseline level of at least one other liver biomarkers has increased; or
(v) the subject baseline level of the second liver biomarker is at least 3 times higher than the upper normal limit of said second liver biomarker or the subject baseline level of second liver biomarker is at least 3 times higher than the subject reference level of the second liver biomarker and the subject baseline level of at least one other liver biomarkers has increased;
or
9) for monitoring a subject's risk of experiencing an adverse side effect caused by lanifibranor treatment, comprising:
a) determining the subject baseline level of a first liver biomarker and the subject baseline level of a second liver biomarker in a baseline sample obtained from the subject, wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof; and
b) comparing the subject baseline level of the first liver biomarker to the upper normal limit of the first liver biomarker and to the subject reference level of the first liver biomarker;
c) comparing the subject baseline level of the second liver biomarker to the upper normal limit of the second liver biomarker and to the subject reference level of the second liver biomarker; and
d) performing close monitoring for a specific period of time if:
a. the subject reference level of the first liver biomarker is below than 1.5 times the upper normal limit of the first liver biomarker, or
b. the subject reference level of the second liver biomarker is below than 1.5 times the upper normal limit of the second liver biomarker
and
c. the subject baseline level of the first liver biomarker is equal or higher than 3 times the upper normal limit of the first liver biomarker but below than 5 times the upper normal limit of the first liver biomarker, or
d. the subject baseline level of the second liver biomarker is equal or higher than 3 times the upper normal limit of the second liver biomarker but below than 5 times the upper normal limit of the second liver biomarker; or
c. the subject baseline level of the first liver biomarker is equal or higher than 5 times the upper normal limit of the first liver biomarker, or
f. the subject baseline level of the second liver biomarker is equal or higher than 5 times the upper normal limit of the second liver biomarker;
wherein the subject is at risk of experiencing an adverse side effect when:
(i) the panel of autoimmune disease biomarkers is present in a baseline sample obtained from the subject, or
(ii) the subject baseline level of the first liver biomarker increases or remains to at least the reference level of the first liver biomarker, or
(iii) the subject baseline level of the second liver biomarker increases or remains to at least the reference level of the second liver biomarker, or
(iv) the subject baseline level of the first liver biomarker is at least 5 times higher than the upper normal limit of the first liver biomarker, or
(v) the subject baseline level of the second liver biomarker is at least 5 times higher than the upper normal limit of the second liver biomarker, or
(vi) the subject baseline level of the first liver biomarker increases or remains to at least 3 times the upper normal limit of the first liver biomarker and the level of two others liver biomarkers has increased, or
(vii) the subject baseline level of the second liver biomarker increases or remains to at least 3 times the upper normal limit of the second liver biomarker and the level of two others liver biomarkers has increased, or
(viii) the subject baseline level of the first liver biomarker increases or remains to at least 3 times the upper normal limit of the first liver biomarker, with the appearance of fatigue, nausea, vomiting, right upper quadrant pain or tenderness, fever, rash, and/or eosinophilia, or
(ix) the subject baseline level of the second liver biomarker increases or remains to at least 3 times the upper normal limit of the second liver biomarker, with the appearance of fatigue, nausea, vomiting, right upper quadrant pain or tenderness, fever, rash, and/or eosinophilia;
or
10) for monitoring a subject's risk of experiencing an adverse side effect caused by lanifibranor treatment, comprising:
a) determining the subject baseline level of a first liver biomarker and a second liver biomarker in a baseline sample obtained from the subject, wherein the first liver biomarker and the second liver biomarker are selected from the group consisting of: aspartate transaminase, alanine transaminase, alkaline phosphatase, gamma-glutamyl transferase, bilirubin, prothrombin time test with an international normalized ratio and a combination thereof;
b) comparing the subject baseline level of the first liver biomarker to the upper normal limit of the first liver biomarker, to the reference level of the first liver biomarker and to the subject reference level of the first liver biomarker;
c) comparing the subject baseline level of the second liver biomarker to the upper normal limit of the second liver biomarker, to the reference level of the second liver biomarker and to the subject reference level of the second liver biomarker; and
d) performing close monitoring for a specific period of time if:
a. the subject reference level of the first liver biomarker is higher than 1.5 times the upper normal limit of the first liver biomarker, or
b. the subject reference level of the second liver biomarker is higher than 1.5 times the upper normal limit of the second liver biomarker
and
c. the subject baseline level of the first liver biomarker is equal or higher than 3 times the subject reference level of the first liver biomarker, or equal or higher than 300 U/L, or
d. the subject baseline level of the second liver biomarker is equal or higher than 3 times the subject reference level of the second liver biomarker, or equal or higher than 300 U/L,
wherein the subject is at risk of experiencing an adverse side effect when:
(i) the panel of autoimmune disease biomarkers is present in a baseline sample obtained from the subject, or
(ii) the subject baseline level of the first liver biomarker increases or remains to at least 3 times the subject reference level of first liver biomarker and higher than 300 U/L, or
(iii) the subject baseline level of the second liver biomarker increases or remains to at least 3 times the subject reference level of second liver biomarker and higher than 300 U/L, or
(iv) the subject baseline level of the first liver biomarker increases or remains to at least 5 times the subject reference level of first liver biomarker, or
(v) the subject baseline level of the second liver biomarker increases or remains to at least 5 times the subject reference level of second liver biomarker, or
(vi) the subject baseline level of the first liver biomarker or the subject baseline level of the second liver biomarker increases or remains equal or higher than 500 U/L, or
(vii) the subject baseline level of the first liver biomarker increases or remains to at least 2 times the subject reference level of the first liver biomarker and the level of at least one other liver biomarker has increased in the baseline sample, or
(viii) the subject baseline level of the second liver biomarker increases or remains to at least 2 times the subject reference level of second liver biomarker and the level of at least one other liver biomarker has increased in the baseline sample, or
(ix) the subject baseline level of the first liver biomarker or the subject baseline level of the second liver biomarker increases or remains equal or higher than 300 U/L, or
(x) the subject baseline level of the first liver biomarker increases or remains to at least 2 times the subject reference level of first liver biomarker, with the appearance of fatigue, nausea, vomiting, right upper quadrant pain or tenderness, fever, rash, and/or eosinophilia, or
(xi) the subject baseline level of the second liver biomarker increases or remains to at least 2 times the subject reference level of second liver biomarker, with the appearance of fatigue, nausea, vomiting, right upper quadrant pain or tenderness, fever, rash, and/or eosinophilia, or
(xii) the subject baseline level of the first liver biomarker or the subject baseline level of the second liver biomarker increases or remains equal or higher than 300 U/L, with the appearance of fatigue, nausea, vomiting, right upper quadrant pain or tenderness, fever, rash, and/or eosinophilia.

2. The method according to claim 1, which is of method 1), wherein the method further comprises administering an effective amount of lanifibranor to the subject selected for treatment with lanifibranor.

3. (canceled)

4. The method according to claim 1, which is of method 2), wherein the method further comprises not administering lanifibranor to said subject.

5-6. (canceled)

7. The method of claim 1, which is of method 4), wherein if the subject is not at risk of experiencing an adverse side effect, the method further comprises administering lanifibranor to the subject.

8-13. (canceled)

14. The method according to claim 1, which is of method 1), 2), 3), 4), 5), 6), 7), 8), 9) or 10), wherein the panel of autoimmune disease biomarkers is indicative for predisposition to an autoimmune disease.

15. The method according to claim 1, which is of method 1), 2), 3), 4), 5), 6), 7), 8), 9) or 10), wherein the panel of autoimmune disease biomarkers comprises at least one autoimmune disease biomarkers selected from the group consisting of: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, anti-liver cytosol type 1 antibody and a combination thereof.

16. The method according to claim 15, wherein the panel of autoimmune disease biomarkers is a combination of the following autoimmune disease biomarkers: anti-thyroid peroxidase antibody, anti-thyroglobulin antibody, anti-thyroid-stimulating hormone (TSH) receptor antibody, anti-nuclear antibody, anti-mitochondrial antibody, anti-smooth muscle antibody, anti-liver kidney microsomal type 1 antibody, and anti-liver cytosol type 1 antibody.

17. The method according to claim 15, wherein the anti-nuclear antibody comprises a panel of different antibodies selected from the group consisting of: anti-double-stranded DNA (dsDNA) antibody, anti-histone antibody, anti-chromatin antibody, anti-Scl-70 antibody, anti-smith antibody, anti-Sm/RNP antibody, anti-Jo-1 antibody, anti-centromeric protein antibody, anti-SSA-52 antibody, anti-SSA-60 antibody, anti-SSB antibody, anti-RNA polymerase III antibody, anti-Ku antibody, anti-CENP-A antibody, anti-CENP-B antibody, anti-fibrillarin antibody, anti-PM/Scl-100 antibody, anti-Th/To antibody, anti-Sp-100 antibody, anti-PML antibody, anti-NXP-2 antibody, anti-MORC3 antibody, anti-MJ antibody and a combination thereof.

18. The method according to claim 1, which is of method 1), 2), 3), 4), 5), 6), 7), 8), 9) or 10), wherein determining the presence of a panel of autoimmune disease biomarkers and/or determining of the subject baseline level of at least one liver biomarker in a baseline sample obtained from the subject, are made at regular interval of time for a determined duration.

19. The method according to claim 1, which is of method 1), 2), 3), 4), 5), 6), 7), 8), 9) or 10), wherein the reference sample is a sample obtained from the subject prior to the first administration of lanifibranor treatment and wherein the baseline sample is a sample obtained from the same subject after administration of lanifibranor treatment.

20. The method according to claim 1, which is of method 1), 2), 3), 4), 5), 6), 7), 8), 9) or 10), wherein the reference sample and the baseline sample are from the same source and are selected from saliva sample, hair sample, skin sample, tissue sample, sputum sample, blood sample, plasma sample, serum sample, vitreal sample, cerebrospinal fluid sample, urine sample, sperm sample and cells sample.

21. The method according to claim 1, which is of method 1), 2), 3), 4), 5), 6), 7), 8), 9) or 10), wherein the subject is suffering from a liver disease selected from the group consisting of: liver fibrosis, liver steatosis, fatty liver disease, acute decompensation, acute decompensation of cirrhosis, acute on chronic liver failure, acute liver failure, decompensated cirrhosis, compensated cirrhosis, non-alcoholic fatty liver disease, non-alcoholic steatohepatitis, metabolic-associated fatty liver disease, metabolic-associated steatohepatitis, diabetes, primary sclerosing cholangitis, hepatocarcinoma, non-alcoholic steatohepatitis with moderate to advanced liver fibrosis, metabolic-associated steatohepatitis with moderate to advanced liver fibrosis, non-cirrhotic non-alcoholic steatohepatitis with moderate to advanced liver fibrosis, non-cirrhotic metabolic-associated steatohepatitis with moderate to advanced liver fibrosis and liver.

22. The method according to claim 21, wherein the liver disease is associated with diabetes.

23. The method according to claim 1, which is of method 1), 2), 3), 4), 5), 6), 7), 8), 9) or 10), wherein the autoimmune disease is selected from the group consisting of: autoimmune hepatitis, dermatomyositis, juvenile idiopathic arthritis, mixed connective tissue disease, primary biliary cholangitis, polymyositis, systemic autoimmune rheumatic disease, Sjögren syndrome, systemic lupus erythematosus, systemic sclerosis, undifferentiated connective tissue disease, autoimmune liver disease and autoimmune thyroid disease.

24. The method according to claim 23, wherein the autoimmune liver disease is selected from the group consisting of: autoimmune hepatitis, autoimmune cholangitis, autoimmune cholangiopathy and overlap syndrome.

25. The method according to claim 2, wherein the effective amount of lanifibranor comprises from 0.5 to 1200 mg of lanifibranor per day.
